(19) United States
(12) Patent Application Publication
(10) Pub. No.: US 2013/0315829 A1
(43) Pub. Date: Nov. 28, 2013

(54) Compounds and Methods for Detecting Reactive Oxygen Species

(71) Applicants: Christopher J. Chang, Berkeley, CA (US); Genevieve C. van de Bittner, Berkeley, CA (US); Elena A. Dubikovskaya, Richmond, CA (US); Carolyn R. Bertozzi, Berkeley, CA (US)

(72) Inventors: Christopher J. Chang, Berkeley, CA (US); Genevieve C. van de Bittner, Berkeley, CA (US); Elena A. Dubikovskaya, Richmond, CA (US); Carolyn R. Bertozzi, Berkeley, CA (US)

(73) Assignee: The Regents of the University of California, Berkeley, CA (US)

(21) Appl. No.: 13/642,082

(22) PCT Filed: Apr. 21, 2011
(86) PCT No.: PCT/US11/33472

§ 371 Date: Jul. 17, 2013

Related U.S. Application Data
(60) Provisional application No. 61/327,428, filed on Apr. 23, 2010.

(51) Int. Cl. A61K 49/00; G01N 21/64

(52) U.S. Cl. CPC A61K 49/00; G01N 21/6486
USPC 424/9.1; 435/29; 436/135; 435/27; 544/229; 548/110

(57) ABSTRACT

The present disclosure provides compounds that detect reactive oxygen species in a living cell, in a multicellular organism, or in a cell-free sample. The compounds find use in a variety of applications, which are also provided. The present disclosure provides compositions comprising a subject compound.

### CROSS-REFERENCE

This application claims the benefit of U.S. Provisional Patent Application No. 61/327,428, filed Apr. 23, 2010, which application is incorporated herein by reference in its entirety.

### STATEMENT REGARDING FEDERALLY SPONSORED RESEARCH

This invention was made with government support under Grant No. GM 79465 awarded by the National Institutes of Health. The government has certain rights in the invention.

### BACKGROUND

Reactive oxygen species (ROS) are involved in various cell signaling pathways that are necessary for cell growth and survival. Correlations have been shown between misregulation of ROS and various diseases, including cancer, diabetes, heart disease, and neurodegenerative diseases. Hydrogen peroxide has been a focus of research geared toward understanding ROS in health and disease because it is a relatively long-lived ROS; thus, it is able to travel through a cell or even across cell membranes before it reacts with a target biomolecule.

Various efforts have been made to detect hydrogen peroxide. For example, fluorescent scaffolds with boronate detection groups have been synthesized for the examination of hydrogen peroxide in cellular systems. Chemiluminescent probes based on peroxalate nanoparticles or on luminol, have been developed.

There remains a need in the field for compounds and methods of detecting ROS, including hydrogen peroxide.

### LITERATURE

WO 2007/050810; WO 2009/152102; Lee et al. (2007) Nat. Mater. 6765; Gross et al. (2009) Nat. Med. 15:455; Kielland et al. (2009) Free Radical Biol. Med. 47:760.

### SUMMARY OF THE INVENTION

The present disclosure provides compounds that detect reactive oxygen species in a living cell, in a multicellular organism, or in a cell-free sample. The compounds find use in a variety of applications, which are also provided. The present disclosure provides compositions comprising a subject compound.

### BRIEF DESCRIPTION OF THE DRAWINGS

FIG. 1 depicts a design strategy for H₂O₂-mediated release of firefly luciferin from peroxy caged luciferin-1 (PCL-1).

FIGS. 2A and 2B depict selective and concentration dependent bioluminescent detection of H₂O₂ by PCL-1.

FIGS. 3A and 3B depict bioluminescent signal from PCL-1 added to LNCaP-luc cells.

FIGS. 4A-D depict bioluminescent signal from PCL-1 in FVB-luc mice.

FIGS. 5A-D depict bioluminescent signal from SHO mice with LNCaP-luc tumors.

### DEFINITIONS

The term “physiological conditions” is meant to encompass those conditions compatible with living cells, e.g., predominantly aqueous conditions of a temperature, pH, salinity, etc. that are compatible with living cells.

“Luciferase” refers to an enzyme that oxidizes a corresponding luciferin, thereby causing bioluminescence. Luciferase enzymes can be found in bacteria, fireflies, fish, squid, dinoflagellates, and other organisms capable of bioluminescence.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and is free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intratracheal and the like. In some embodiments the composition is suitable for administration by a transdermal route, using a penetration enhancer other than dimethylsulfoxide (DMSO). In other embodiments, the pharmaceutical compositions are suitable for administration by a route other than transdermal administration. A pharmaceutical composition will in some embodiments include a subject compound and a pharmaceutically acceptable excipient. In some embodiments, a pharmaceutically acceptable excipient is other than DMSO.

As used herein, “pharmaceutically acceptable derivatives” of a compound of the invention include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and are either pharmaceutically active or are prodrugs.

A “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

A “pharmaceutically acceptable ester” of a compound of the invention means an ester that is pharmaceutically acceptable and that possesses the desired pharmacological activity of the parent compound, and includes, but is not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids.

A “pharmaceutically acceptable enol ether” of a compound of the invention means an enol ether that is pharmaceutically acceptable and that possesses the desired pharmacological activity of the parent compound, and includes, but is not limited to, derivatives of formula C═C(OR) where R is hydrogen, alkyl, alkenyl, alkynyl, aryl, heteroaryl, aralkyl, heteroaralkyl, cycloalkyl or heterocyclyl.

A “pharmaceutically acceptable solvate or hydrate” of a compound of the invention means a solvate or hydrate complex that is pharmaceutically acceptable and that possesses the desired pharmacological activity of the parent compound, and includes, but is not limited to, complexes of a compound of the invention with one or more solvent or water molecules, or 1 to about 100, or 1 to about 10, or one to about 2, 3 or 4, solvent or water molecules.

“Pro-drugs” means any compound that releases an active parent drug according to one or more of the generic formulas shown below in vivo when such prodrug is administered to a mammalian subject. Prodrugs of a compound of one or more of the generic formulas shown below are prepared by modifying functional groups present in the compound of the generic formula in such a way that the modifications may be cleaved in vivo to release the parent compound. Prodrugs include compounds of one or more of the generic formulas shown below wherein a hydroxy, amino, or sulfhydryl group in one or more of the generic formulas shown below is bonded to any group that may be cleaved in vivo to regenerate the free hydroxyl, amino, or sulfhydryl group, respectively. Examples of prodrugs include, but are not limited to esters (e.g., acetate, formate, and benzoate derivatives), carbamates (e.g., N,N-dimethylaminocarbonyl) of hydroxy functional groups in compounds of one or more of the generic formulas shown below, and the like.

The term “organic group” and “organic radical” as used herein means any carbon-containing group, including hydrocarbon groups that are classified as an aliphatic group, cyclic group, aromatic group, functionalized derivatives thereof and/or various combinations thereof. The term “aliphatic group” means a saturated or unsaturated linear or branched hydrocarbon group and encompasses alkyl, alkenyl, and alkynyl groups, for example. The term “alkyl group” means a substituted or unsubstituted, saturated linear or branched hydrocarbon group or chain (e.g., C₁ to C₈) including, for example, methyl, ethyl, isopropyl, tert-butyl, heptyl, iso-propyl, n-octyl, dodecyl, octadecyl, amyl, 2-ethylhexyl, and the like. Suitable substituents include carboxy, protected carboxy, amino, protected amino, halo, hydroxy, protected hydroxy, nitro, cyano, monosubstituted amino, protected monosubstituted amino, disubstituted amino, C₁ to C₇ alkoxy, C₁ to C₇ acyl, C₁ to C₇ acyloxy, and the like. The term “substituted alkyl” means the above defined alkyl group substituted from one to three times by a hydroxy, protected hydroxy, amino, protected amino, cyano, halo, trifloromethyl, mono-substituted amino, di-substituted amino, lower alkoxy, lower alkylthio, carboxy, protected carboxy, or a carboxy, amino, and/or hydroxy salt. As used in conjunction with the substituents for the heteroaryl rings, the terms “substituted (cycloalkyl)alkyl” and “substituted cycloalkyl” are as defined below substituted with the same groups as listed for a “substituted alkyl” group. The term “alkenyl group” means an unsaturated, linear or branched hydrocarbon group with one or more carbon-carbon double bonds, such as a vinyl group. The term “alkynyl group” means an unsaturated, linear or branched hydrocarbon group with one or more carbon-carbon triple bonds. The term “cyclic group” means a closed ring hydrocarbon group that is classified as an alicyclic group, aromatic group, or heterocyclic group. The term “alicyclic group” means a cyclic hydrocarbon group having properties resembling those of aliphatic groups. The term “aromatic group” or “aryl group” means a mono- or polycyclic aromatic hydrocarbon group, and may include one or more heteroatoms, and which are further defined below. The term “heterocyclic group” means a closed ring hydrocarbon in which one or more of the atoms in the ring are an element other than carbon (e.g., nitrogen, oxygen, sulfur, etc.), and are further defined below.

“Organic groups” may be functionalized or otherwise comprise additional functionalities associated with the organic group, such as carboxyl, amino, hydroxyl, and the like, which may be protected or unprotected. For example, the phrase “alkyl group” is intended to include not only pure open chain saturated hydrocarbon alkyl substituents, such as methyl, ethyl, propyl, t-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ethers, esters, haloalkyls, nitroalkyls, carboxyalkyls, hydroxyalkyls, sulfoalkyls, etc.

The terms “halo” and “halogen” refer to the fluoro, chloro, bromo or iodo groups. There can be one or more halogen, which are the same or different. Halogens of particular interest include chloro and bromo groups.

The term “haloalkyl” refers to an alkyl group as defined above that is substituted by one or more halogen atoms. The halogen atoms may be the same or different. The term “dihaloalkyl” refers to an alkyl group as described above that is substituted by two halo groups, which may be the same or different. The term “trihaloalkyl” refers to an alkyl group as describe above that is substituted by three halo groups, which may be the same or different. The term “perhaloalkyl” refers to a haloalkyl group as defined above wherein each hydrogen atom in the alkyl group has been replaced by a halogen atom. The term “perfluoroalkyl” refers to a haloalkyl group as defined above wherein each hydrogen atom in the alkyl group has been replaced by a fluoro group.

The term “cycloalkyl” means a mono-, bi-, or tricyclic saturated ring that is fully saturated or partially unsaturated. Examples of such a group included cyclopropyl, cyclobutyl, cyclopentyl, cyclohexyl, cycloheptyl, adamantyl, cyclooctyl, cis- or trans decalin, bicyclo[2.2.1]hept-2-ene, cyclohex-1-enyl, cyclopent-1-enyl, 1,4-cyclooctadienyl, and the like.

The term “(cycloalkyl)alkyl” means the above-defined alkyl group substituted for one of the above cycloalkyl rings. Examples of such a group include (cyclohexyl)methyl, 3-(cyclopropyl)-n-propyl, 5-(cyclopentyl)hexyl, 6-(adamantyl)hexyl, and the like.

The term “substituted phenyl” specifies a phenyl group substituted with one or more moieties, and in some instances one, two, or three moieties, chosen from the groups consisting of halogen, hydroxy, protected hydroxy, cyano, nitro, trifluoromethyl, C₁ to C₇ alkyl, C₁ to C₇ alkoxy, C₁ to C₇ acyl, C₁ to C₇ acyloxy, carboxy, oxycarboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₆ alkyl)carboxamide, protected N—(C₁ to C₆ alkyl)carboxamide, N,N-di(C₁ to C₆ alkyl)carboxamide, trifluoromethyl, N—((C₁ to C₆ alkyl)sulfonyl)amino, N-(phenylsulfonyl)amino or phenyl, substituted or unsubstituted, such that, for example, a biphenyl or naphthyl group results.

Examples of the term “substituted phenyl” includes a mono- or di(halo)phenyl group such as 2, 3 or 4-chlorophenyl, 2,6-dichlorophenyl, 2,5-dichlorophenyl, 3,4-dichlorophenyl, 2, 3 or 4-bromophenyl, 3,4-dibromophenyl, 3-chloro-4-fluorophenyl, 2, 3 or 4-fluorophenyl and the like; a mono or di(hydroxy)phenyl group such as 2, 3, or 4-hydroxyphenyl, 2,4-dihydroxyphenyl, the protected-hydroxy derivatives thereof and the like; a nitrophenyl group such as 2, 3, or 4-nitrophenyl; a cyanophenyl group, for example, 2, 3 or 4-cyanophenyl; a mono- or di(alkyl)phenyl group such as 2, 3, or 4-methylphenyl, 2,4-dimethylphenyl, 2, 3 or 4-(iso-propyl)phenyl, 2, 3, or 4-ethylphenyl, 2, 3 or 4-(n-propyl)phenyl and the like; a mono or di(alkoxy)phenyl group, for example, 2,6-dimethoxyphenyl, 2, 3 or 4-(isopropoxy)phenyl, 2, 3 or 4-(t-butoxy)phenyl, 3-ethoxy-4-methoxyphenyl and the like; 2, 3 or 4-trifluoromethylphenyl; a mono- or dicarboxyphenyl or (protected carboxy)phenyl group such as 2, 3 or 4-carboxyphenyl or 2,4-di(protected carboxy)phenyl; a mono- or di(hydroxymethyl)phenyl or (protected hydroxymethyl)phenyl such as 2, 3 or 4-(protected hydroxymethyl)phenyl or 3,4-di(hydroxymethyl)phenyl; a mono- or di(aminomethyl)phenyl or (protected aminomethyl)phenyl such as 2, 3 or 4-(aminomethyl)phenyl or 2,4-(protected aminomethyl)phenyl; or a mono- or di(N-(methylsulfonylamino))phenyl such as 2, 3 or 4-(N-(methylsulfonylamino))phenyl. Also, the term “substituted phenyl” represents disubstituted phenyl groups wherein the substituents are different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy-4-chlorophenyl, and the like.

The term “(substituted phenyl)alkyl” means one of the above substituted phenyl groups attached to one of the above-described alkyl groups. Examples of include such groups as 2-phenyl-1-chloroethyl, 2-(4′-methoxyphenyl)ethyl, 4-(2′,6′-dihydroxy phenyl)n-hexyl, 2-(5′-cyano-3′-methoxyphenyl)n-pentyl, 3-(2′,6′-dimethylphenyl)n-propyl, 4-chloro-3-aminobenzyl, 6-(4′-methoxyphenyl)-3-carboxy(n-hexyl), 5-(4′-aminomethylphenyl)-3-(aminomethyl)n-pentyl, 5-phenyl-3-oxo-n-pent-1-yl, (4-hydroxynapth-2-yl)methyl and the like.

As noted above, the term “aromatic” or “aryl” refers to six membered carbocyclic rings. Also as noted above, the term “heteroaryl” denotes optionally substituted five-membered or six-membered rings that have 1 to 4 heteroatoms, such as oxygen, sulfur and/or nitrogen atoms, in particular nitrogen, either alone or in conjunction with sulfur or oxygen ring atoms.

Furthermore, the above optionally substituted five-membered or six-membered rings can optionally be fused to an aromatic 5-membered or 6-membered ring system. For example, the rings can be optionally fused to an aromatic 5-membered or 6-membered ring system such as a pyridine or a triazole system, and preferably to a benzene ring.

The following ring systems are examples of the heterocyclic (whether substituted or unsubstituted) radicals denoted by the term “heteroaryl”: thienyl, furyl, pyrrolyl, pyrrolidinyl, imidazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, oxazinyl, triazinyl, thiadiazinyl tetrazolo, 1,5-[b]pyridazinyl and purinyl, as well as benzo-fused derivatives, for example, benzoxazolyl, benzthiazolyl, benzimidazolyl and indolyl.

Substituents for the above optionally substituted heteroaryl rings are from one to three halo, trihalomethyl, amino, protected amino, amino salts, mono-substituted amino, di-substituted amino, carboxy, protected carboxy, carboxylate salts, hydroxy, protected hydroxy, salts of a hydroxy group, lower alkoxy, lower alkylthio, alkyl, substituted alkyl, cycloalkyl, substituted cycloalkyl, (cycloalkyl)alkyl, substituted (cycloalkyl)alkyl, phenyl, substituted phenyl, phenylalkyl, and (substituted phenyl)alkyl. Substituents for the heteroaryl group are as heretofore defined, or in the case of trihalomethyl, can be trifluoromethyl, trichloromethyl, tribromomethyl, or triiodomethyl. As used in conjunction with the above substituents for heteroaryl rings, “lower alkoxy” means a C₁ to C₄ alkoxy group, similarly, “lower alkylthio” means a C₁ to C₄ alkylthio group.

The term “(monosubstituted)amino” refers to an amino group with one substituent chosen from the group consisting of phenyl, substituted phenyl, alkyl, substituted alkyl, C₁ to C₄ acyl, C₂ to C₇ alkenyl, C₂ to C₇ substituted alkenyl, C₂ to C₇ alkyllyl, C₇ to C₁₆ alkylaryl, C₇ to C₁₆ substituted alkylaryl and heteroaryl group. The (monosubstituted) amino can additionally have an amino-protecting group as encompassed by the term “protected (monosubstituted)amino.” The term “(disubstituted)amino” refers to amino groups with two substituents chosen from the group consisting of phenyl, substituted phenyl, alkyl, substituted alkyl, C₁ to C₇ acyl, C₂ to C₇ alkenyl, C₂ to C₇ alkynyl, C₇ to C₁₆ alkylaryl, C₇ to C₁₆ substituted alkylaryl and heteroaryl. The two substituents can be the same or different.

The term “heteroaryl(alkyl)” denotes an alkyl group as defined above, substituted at any position by a heteroaryl group, as above defined.

“Optional” or “optionally” means that the subsequently described event, circumstance, feature, or element may, but need not, occur, and that the description includes instances where the event or circumstance occurs and instances in which it does not. For example, “heterocyclo group optionally mono- or di-substituted with an alkyl group” means that the alkyl may, but need not, be present, and the description includes situations where the heterocyclo group is mono- or disubstituted with an alkyl group and situations where the heterocyclo group is not substituted with the alkyl group.

“Substituted” refers to a group in which one or more hydrogen atoms are independently replaced with the same or different substituent(s). Typical substituents include, but are not limited to, alkylenedioxy (such as methylenedioxy), -M, —R⁶⁰, —O⁻, ═O, —OR⁶⁰, —SR⁶⁰, —S⁻, ═S, —NR⁶⁰R⁶¹, ═NR⁶⁰, —CF₃, —CN, —OCN, —SCN, —NO, —NO₂, ═N₂, —N₃, —S(O)₂O⁻, —S(O)₂OH, —S(O)₂R⁶⁰, —OS(O)₂O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹ and —C(NR⁶²)NR⁶⁰R⁶¹ where M is halogen; R⁶⁰, R⁶¹, R⁶² and R⁶³ are independently hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl or substituted heteroaryl, or optionally R⁶⁰ and R⁶¹ together with the nitrogen atom to which they are bonded form a cycloheteroalkyl or substituted cycloheteroalkyl ring.

As used herein, the term “linker” or “linkage” or “linking group” refers to a linking moiety that connects two groups and has a backbone of 20 atoms or less in length. A linker or linkage may be a covalent bond that connects two groups or a chain of between 1 and 20 atoms in length, for example of about 1, 2, 3, 4, 5, 6, 8, 10, 12, 14, 16, 18 or 20 carbon atoms in length, where the linker may be linear, branched, cyclic or a single atom. In certain cases, one, two, three, four or five or more carbon atoms of a linker backbone may be optionally substituted with a sulfur, nitrogen or oxygen heteroatom. The bonds between backbone atoms may be saturated or unsaturated, usually not more than one, two, or three unsaturated bonds will be present in a linker backbone. The linker may include one or more substituent groups, for example with an alkyl, aryl or alkenyl group. A linker may include, without limitations, oligo(ethylene glycol); ethers, thioethers, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable.

Compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

A subject compound may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see, e.g., the discussion in Chapter 4 of “Advanced Organic Chemistry”, 4th edition J. March, John Wiley and Sons, New York, 1992).

“In combination with” as used herein refers to uses where, for example, the first compound is administered during the entire course of administration of the second compound; where the first compound is administered for a period of time that is overlapping with the administration of the second compound, e.g. where administration of the first compound begins before the administration of the second compound and the administration of the first compound ends before the administration of the second compound ends; where the administration of the second compound begins before the administration of the first compound and the administration of the second compound ends before the administration of the first compound ends; where the administration of the first compound begins before administration of the second compound begins and the administration of the second compound ends before the administration of the first compound ends; where the administration of the second compound begins before administration of the first compound begins and the administration of the first compound ends before the administration of the second compound ends. As such, “in combination” can also refer to regimen involving administration of two or more compounds. “In combination with” as used herein also refers to administration of two or more compounds which may be administered in the same or different formulations, by the same of different routes, and in the same or different dosage form type.

The terms “subject,” “individual,” “host,” and “patient” are used interchangeably herein to a multicellular organism that is a member or members of any mammalian or non-mammalian species. Subjects and patients thus include, without limitation, primate (including humans and non-human primates), canine, feline, ungulate (e.g., equine, bovine, swine (e.g., pig)), avian, and other subjects. Humans are of particular interest in some embodiments. Non-human mammals having commercial importance (e.g., livestock and domesticated animals) are of particular interest in some embodiments. The term “multicellular organism” includes humans, non-human animals, and plants. Where a multicellular organism is an animal (humans and non-human animals), “multicellular organism” can be used interchangeably with “individual.”

“Mammal” refers to a member or members of any mammalian species, and includes, by way of example, canines; felines; equines; bovines; ovines; rodentia, etc. and primates, e.g., humans. Non-human animal models, particularly mammals, e.g. a non-human primate, a murine (e.g., a mouse, a rat), lagomorpha, etc. may be used for experimental investigations.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components. The term “biological sample” encompasses a clinical sample, and also includes cells in culture, cell supernatants, cell lysates, serum, plasma, biological fluid, and tissue samples. In some embodiments, a “biological sample” is cell free.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a compound that detects a reactive oxygen species” includes a plurality of such compounds and reference to “the reactive oxygen species” includes reference to one or more reactive oxygen species and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides compounds that detect reactive oxygen species in a living cell (in vivo, ex vivo, or in vitro), in a multicellular organism, in extracellular fluid, or in a cell-free sample. The compounds find use in a variety of applications, which are also provided. The present disclosure provides compositions comprising a subject compound.

A subject compound can selectively detect a reactive oxygen species (ROS) in a living cell (in vivo, ex vivo, or in vitro), in a multicellular organism, in extracellular fluid, or in a cell-free sample. Upon reaction of a subject compound with a reactive oxygen species, a moiety is released from the compound, where the moiety generates a detectable signal, either directly or through action of another molecule (e.g., after being acted upon by an enzyme).

A subject compound is substantially non-toxic to a living cell, and thus is suitable for detecting an ROS in a living cell (in vivo, ex vivo, or in vitro), in the extracellular medium in which a living cell is cultured in vitro or ex vivo, or extracellularly in a multicellular organism. ROS include oxygen related free radicals such as superoxide (O₂⁻), peroxyl (ROO⁻), alkoxyl (RO⁻), hydroxyl (HO⁻), and nitric oxide; and non-radical species, such as hydrogen peroxide (H₂O₂), hypochlorous acid, singlet oxygen, alkoxides, hydroxide, and peroxynitrite.

A subject compound can provide for detection of an ROS (e.g., hydrogen peroxide) in a living cell (in vivo, ex vivo, or in vitro), in a multicellular organism, in extracellular fluid (e.g., in the extracellular fluid of an in vitro cell), or in a cell-free sample, where the ROS is present in the living cell (in vivo, ex vivo, or in vitro), in a multicellular organism (e.g., in an extracellular fluid in a multicellular organism), or in a cell-free sample, at a concentration of from about 100 μM to about 50 μM, from about 50 μM to about 25 μM, from about 25 μM to about 10 μM, from about 10 μM to about 1 μM, from about 1 μM to about 100 nM, from about 100 nM to about 50 nM, from about 50 nM to about 25 nM, from about 25 nM to about 10 nM, or from about 10 nM to about 1 nM. In some embodiments, a subject compound can provide for detection of H₂O₂ in a living cell (in vivo or in vitro), in a multicellular organism, or in a cell-free sample in a range of from about 2.5 μM to about 250 μM, or in a range of from about 50 μM to about 250 μM.

A subject compound can provide for detection of an ROS in a living cell (in vivo or in vitro), in a multicellular organism, or in a cell-free sample, where the ROS is present in the living cell (in vivo or in vitro), in the multicellular organism (e.g., in an extracellular fluid in the multicellular organism), or in a cell-free sample at a concentration of from about 1000 μM to about 2.5 μM.

In some embodiments, a subject compound provides for selective detection of a particular ROS. For example, in some embodiments, a subject compound selectively reacts with hydrogen peroxide, compared to other ROS. In some embodiments, a subject compound reacts with hydrogen peroxide, and does not substantially react with ROS other than hydrogen peroxide, e.g., the compound does not substantially react with any of superoxide anion, nitric oxide, peroxyl radical, alkoxyl radical, hydroxyl radical, hypochlorous acid, and singlet oxygen.

A subject compound, upon reaction with an ROS (e.g., hydrogen peroxide), produces photons of a wavelength that can penetrate tissues; as such, a subject compound can provide information regarding ROS concentration (e.g., H₂O₂ concentration) in a living animal. For example, in some embodiments, a subject compound, upon reaction with an ROS, produces photons longer than 430 nm, e.g., in some embodiments, a subject compound, upon reaction with an ROS, produces photons having a wavelength in a range of from about 450 nm to about 500 nm, from about 500 nm to about 550 nm, from about 550 nm to about 600 nm, from about 600 nm to about 650 nm, from about 650 nm to about 700 nm, or greater than 700 nm.

A subject compound, upon reaction with an ROS, produces photons of a wavelength that can penetrate tissues; as such, a subject compound can provide information regarding ROS concentration (e.g., H₂O₂ concentration) in a living animal. For example, in some embodiments, a subject compound, upon reaction with an ROS, produces photons longer than 400 nm, e.g., in some embodiments, a subject compound, upon reaction with an ROS, produces photons having a wavelength in a range of from about 400 nm to about 550 nm (e.g., with renilla luciferase and other marine luciferases), from about 500 nm to about 650 nm (e.g., with firefly luciferase), from about 550 nm to about 680 nm (e.g., with red-shifted firefly luciferases and click beetle luciferase).

In certain embodiments, a subject compound, upon reaction with an ROS, produces photons a range of from about 560 nm to about 660 nm (e.g., with red-shifted firefly luciferases and click beetle luciferase).

### Compounds

The present disclosure provides compounds that provide for the selective detection of reactive oxygen species such as H₂O₂ in a living cell (in vivo, ex vivo, or in vitro), in a multicellular organism, in extracellular fluid, or in a cell-free sample.

The subject compounds are self-immolative, e.g., compounds that respond to an external stimulus (e.g., a reactive oxygen species) to undergo a fragmentation or cleavage to release a detectable moiety. In some embodiments, the subject compounds include an ROS— (e.g., a H₂O₂—) sensitive aryl or heteroaryl boronate group that is connected to detectable moiety via a cleavable linker. The aryl or heteroaryl boronate group is conjugated to a cleavable bond of the cleavable linker, such that upon an oxidation reaction of the aryl or heteroaryl boronate (e.g., after reaction with an ROS such as H₂O₂), electrons can be donated or resonate through the conjugated system to spontaneously cleave the cleavable bond of the linker and release a leaving group connected to the detectable moiety.

The disclosure provides a compound of formula (I):

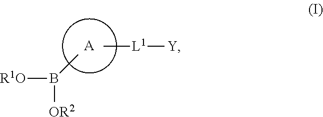

wherein
R¹ and R² are selected from hydrogen and alkyl; or R¹ and R² together form a boronic ester ring or substituted boronic ester ring;
A ring is selected from aryl, substituted aryl, heteroaryl, and substituted heteroaryl;
L¹ is cleavable linker group that provides for release of Y upon reaction of the —B(OR¹)(OR²) group with a reactive oxygen species; and
Y is a detectable moiety that is released upon reaction of the compound with a reactive oxygen species; wherein, after release, the detectable moiety generates a detectable signal, either directly (e.g., by fluorescence or luminescence) or indirectly (e.g., after an enzyme mediated reaction).

In formula (I), R¹ and R² can be selected from hydrogen and alkyl; or R¹ and R² together can form a boronic ester ring or substituted boronic ester ring. In certain instances, both R¹ and R² are hydrogen. In certain instances, both R¹ and R² are alkyl, such as, for example, methyl, ethyl, propyl, isopropyl, and butyl. In certain instances, R¹ and R² together form a boronic ester ring or substituted boronic ester ring. In certain instances, R¹ and R² together form a boronic ester ring. In certain instances, R¹ and R² together form a substituted boronic ester ring. In certain instances, the —B(OR¹)(OR²) group is selected from the following:

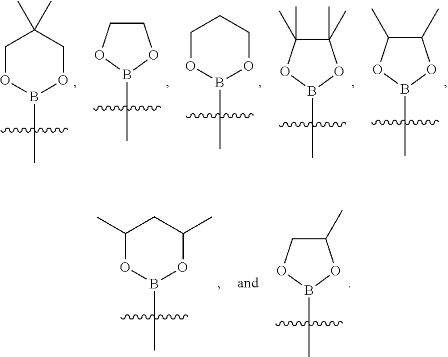

In formula (I), the A ring can be selected from aryl, substituted aryl, heteroaryl, and substituted heteroaryl. In certain instances, the A ring is aryl. In certain instances, the A ring is substituted aryl. In certain instances, the A ring is phenyl. In certain instances, the A ring is substituted phenyl. In certain instances, the A ring is heteroaryl. In certain instances, the A ring is substituted heteroaryl. In certain instances, the A ring is pyridinyl. In certain instances, the A ring is substituted pyridinyl. The A ring connects the —B(OR¹)(OR²) group and L¹. The arrangement of these groups on the A ring is at any suitable ring positions that provides for electronic communication between the two groups (e.g., delocalization of a lone pair of electrons from one group to the other). For example, when A is a phenyl ring, arrangement of the —B(OR¹)(OR²) group and L¹ group either ortho- or para- to each other provides for delocalization of a lone pair of electrons from the site of —B(OR¹)(OR²) group oxidation to the cleavable bond of the cleavable linker.

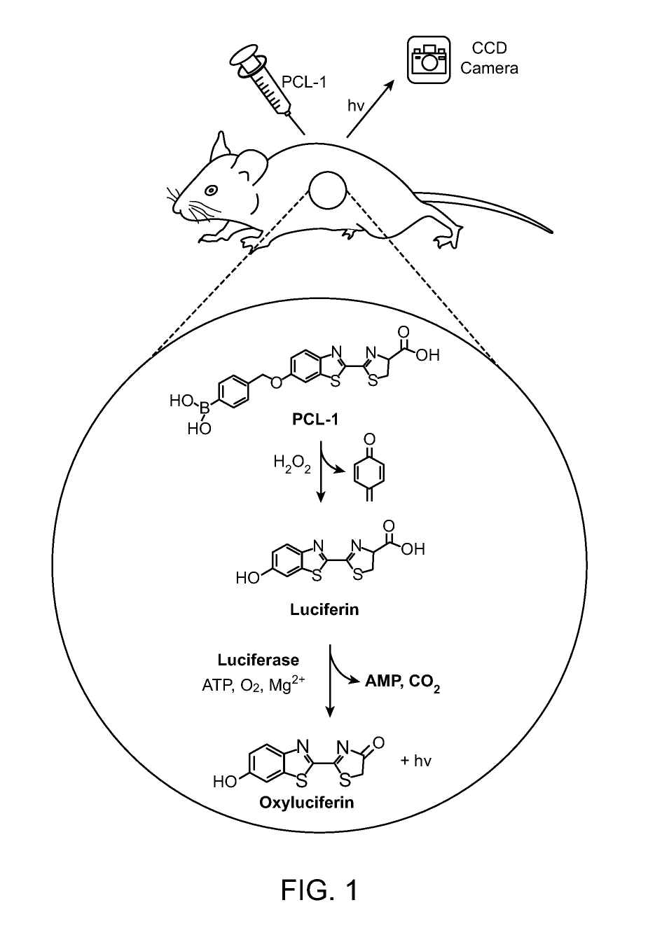

As used herein, the term “cleavable linker group” refers to a linker that can be selectively cleaved to produce two products. Application of suitable cleavage conditions to a molecule containing a cleavable linker that is cleaved by the cleavage conditions will produce two byproducts. A cleavable linker of the present invention is stable, e.g. to physiological conditions, until the molecule is contacted with a cleavage-inducing stimulus, such as a cleavage-inducing agent (e.g., a reactive oxygen species). Exemplary conditions are set forth below and are depicted in the exemplary compound and scheme of FIG. 1. In formula (I), L¹ is cleavable linker group that provides for release of Y upon reaction of the —B(OR¹)(OR²) group with a reactive oxygen species, where release of Y includes cleavage of a cleavable bond to release a leaving group. For example, upon reaction (e.g., a hydroboration-oxidation reaction) of the aryl or heteroaryl —B(OR¹)(OR²) group with a reactive oxygen species (e.g., H₂O₂), the cleavable bond of the cleavable linking group L¹ is spontaneously cleaved to release the leaving group and the detectable moiety Y. The cleavable bond connects the leaving group to an adjacent carbon atom that is conjugated to the aryl boronate group that is oxidized. A cascade occurs in which an electron pair is donated from the site of oxidation through the aryl or heteroaryl group to the carbon atom adjacent to the leaving group of the linker, thereby cleaving the cleavable bond. The L¹ linker group provides for release of Y by fragmentation or cleavage of the linker with the donation of the electron pair. The L¹ linker group comprises segments of atoms, in which the segments can be displaced into two byproducts after a cleavage-inducing stimulus (e.g., reaction of the —B(OR¹)(OR²) group with a reactive oxygen species).

The L¹ linker group can include one or more groups such as, but not limited to, alkyl, ether, carbamate, carbonate, carbamide (urea), ester, thioester, aryl, amide, imines, phosphate esters, hydrazones, acetals, orthoesters, and combinations thereof. In some embodiments, the L¹ linker group is described the following structure:

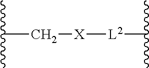

where X is a leaving group and L² is a linking group, wherein the bond that connects X to the adjacent —CH₂— group (e.g., CH₂—X) is a cleavable bond. In some embodiments X is oxygen or sulfur. In some embodiments, the leaving group is a carbamate, a carbonate, a thiol, an alcohol, an amino (e.g., an aryl amino) or a phenol group.

In certain embodiments, the linking group L² is a covalent bond or a chain of between 1 and 12 atoms in length (e.g., between 1 and 10, 1 and 8, 1 and 6 or 1 and 4 atoms in length). In some cases, L² is a chain of between 1 and 12 atoms in length that further includes a second leaving group adjacent to the detectable moiety Y (e.g., L² has a structure L³-X² where L³ is a linking group and X² is the second leaving group, e.g., O, NH or NR where R is an alkyl), such that upon cleavage of the cleavable bond (CH₂—X), a moiety is released (e.g., HX-L³-X²—Y) that includes both the first leaving group (X), L³-X² and Y. In such cases, the released moiety (e.g., HX-L³-X²—Y) may undergo further cleavage or fragmentation (e.g., via an intramolecular cyclization-release) to release HX²—Y, a moiety that may be directly or indirectly detected (e.g., a luciferin or aminoluciferin). In some embodiments, L² is a covalent bond, such that upon cleavage of the cleavable bond (CH₂—X), a moiety is released (e.g., HX—Y) that includes both the leaving group X and the detectable moiety Y, that together may be directly or indirectly detected. When referring to the detectable moiety that is released (e.g., a luciferin moiety) it is understood that the leaving group (X and/or X²) and segments of the linker may be attached to the detectable moiety being described. It is understood that in any of the embodiments described herein that upon cleavage of the cleavable bond of the linker, a moiety is released that may be directly or indirectly detected, or that may undergo further cleavage/fragmentation (e.g., via an intramolecular cyclization-release) prior to being directly or indirectly detected.

In certain instances, the L¹ linker group is selected from the following:

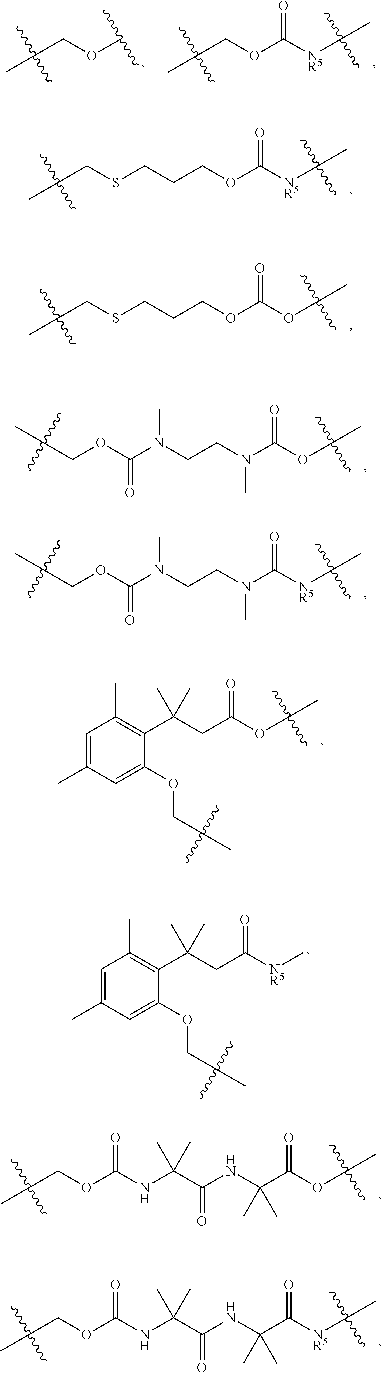

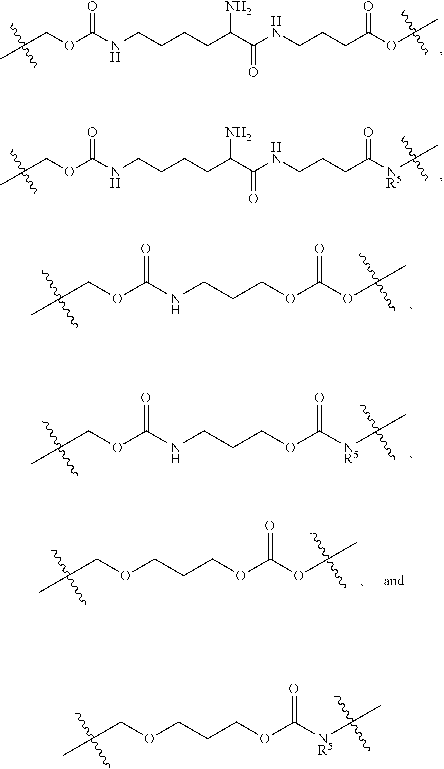

where R⁵ is hydrogen, alkyl, substituted alkyl or alkoxy, where optionally R⁵ may be covalently connected to Y (e.g., to form a fused ring system).

In formula (I), Y comprises a detectable moiety that is released upon reaction of the compound with a reactive oxygen species. After release, the detectable moiety is detected either directly (e.g., Y comprises a luminogenic or fluorogenic moiety that upon release generates a luminescent or fluorescent signal) or indirectly (e.g., Y comprises a detectable moiety that upon release undergoes further reaction (e.g., an enzyme mediated reaction or an activator mediated reaction) to produce a detectable light signal. In some cases, the detectable moiety itself is converted into a light emitting product using an enzyme mediated reaction. Exemplary detectable moieties include, but are not limited to, luciferins and luminogenic or fluorogenic moieties.

In some embodiments, Y comprises a detectable moiety (e.g., a luminophore) that is a luciferin. As used herein, the term “luciferin” refers to a small molecule substrate of luciferase that is oxidized in the presence of the enzyme to produce an oxyluciferin and light energy. Luciferins are characterized by using reactive oxygen species to emit light. Luciferins may be naturally occurring substrates or synthetic analogues thereof. Any suitable substrate of luciferase may be utilized as a luciferin in the present invention. Luciferins of interest include, but are not limited to, a firefly luciferin; an aminoluciferin; a Latia luciferin; a dinoflagellatte luciferin; coelenterazine; a modified coelenterazine as described in U.S. Pat. No. 7,537,912; a coelenterazine analog as described in U.S. Patent Publication No. 2009/0081129 (e.g., a membrane permeant coelenterazine analog as described in U.S. Patent Publication No. 2009/0081129, e.g., one of Structures II, III, IV, V, and VI of U.S. Patent Publication No. 2009/0081129); vargulin; dihydroluciferin; luciferin 6′ methylether; luciferin 6′ chloroethylether. See, e.g., Branchini, B. R. et al. Anal. Biochem. 2010, 396, 290-296; and Mezzanotte, L. et al., In vivo bioluminescence imaging of murine xenograft cancer models with a red-shifted thermostable luciferase. Mol. Imaging. Biol. (2010) 12:406.

In some embodiments, Y comprises an optionally substituted luciferin moiety, where the luciferin moiety is described by one of the following structures:

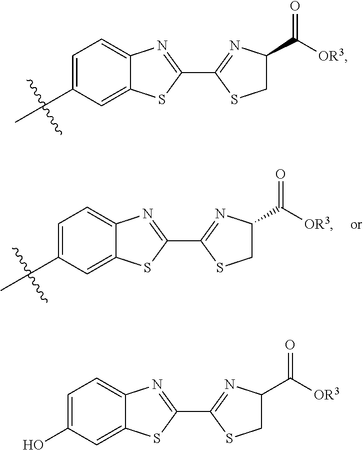

wherein R³ is hydrogen, alkyl or substituted alkyl. The luciferin structure herein is shown with the attachment point to the linker Depending on the atom at the attachment point, upon cleavage of the linker to release Y, Y can include a luciferin moiety or an aminoluciferin moiety described by one of the following structures:

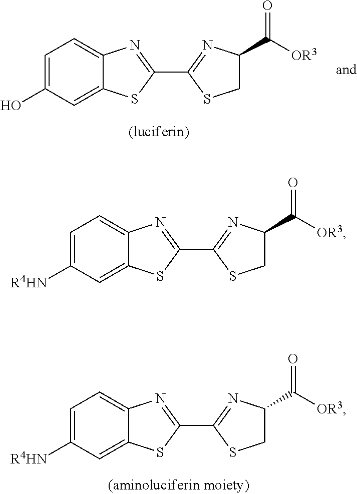

wherein R³ is hydrogen, alkyl or substituted alkyl; and R⁴ is hydrogen, alkyl, substituted alkyl or alkoxy.

In some embodiments, Y is a luciferin or aminoluciferin moiety as shown above that is released directly upon cleavage of the cleavable bond of the linker. In some embodiments, cleavage of the cleavable bond of the linker releases a moiety that includes a luciferin or aminoluciferin moiety as shown in the structures above that is connected to a leaving group via a linking group (e.g., HX-L²-Y, where Y is luciferin or aminoluciferin, L² is a linking group and HX includes the leaving group). In such cases, the released moiety (e.g., HX-L²-Y, where Y is luciferin or aminoluciferin) may undergo a further intramolecular cyclization-release reaction to release a moiety Y (e.g., luciferin or aminoluciferin) suitable for detection (e.g., using luciferase).

In some embodiments, Y comprises an optionally substituted coelenterazine moiety, where coelenterazine has the structure:

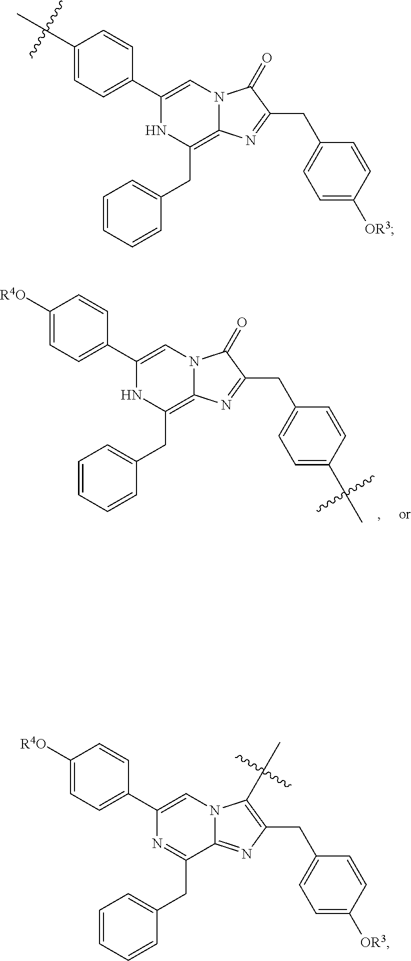

wherein R³ and R⁴ are each independently selected from hydrogen, acyl, acyloxy, and acylamino. The coelenterazine structures herein are shown with different attachment points to the linker Depending on the atom at the attachment point, upon cleavage of the linker to release Y, it is understood that the released moiety may include a leaving group X and/or X2 attached to the structures shown herein.

In some embodiments, Y comprises a compound of the formula:

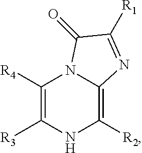

wherein R¹, R², R³, and R⁴ can be independently H, alkyl, heteroalkyl, aryl, or combinations thereof. The structure can be attached to the linker as a substituent on the core rings or as a substituent on any of R¹, R², R³, and R⁴. In the above structure, the core ring structure can be optionally substituted. The structure is described in U.S. Pat. No. 7,537,912. In some embodiments, Y comprises a modified coelenterazine as described in U.S. Pat. No. 7,537,912, which is herein incorporated by reference in its entirety.

In some embodiments, Y comprises an optionally substituted membrane-permeant coelenterazine moiety of the formula:

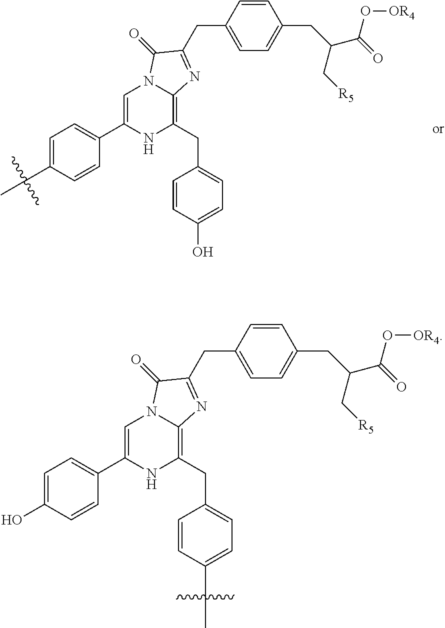

wherein R₄ and R₅ may independently be alkyl or aralkyl, and R₄ may be aryl or optionally substituted aryl, aralkyl or optionally substituted aralkyl, and R₅ may be alkyl, optionally substituted alkyl, alkoxy, aralkyl, or optionally substituted aralkyl, aryl, or a heterocycle. The structures herein are shown with attachment points to the linker Depending on the atom at the attachment point, upon cleavage of the linker to release Y, it is understood that the released moiety may include a leaving group X and/or X2 attached to any one of the structures shown herein.

In some embodiments, Y comprises an optionally substituted membrane-permeant coelenterazine moiety of the formula:

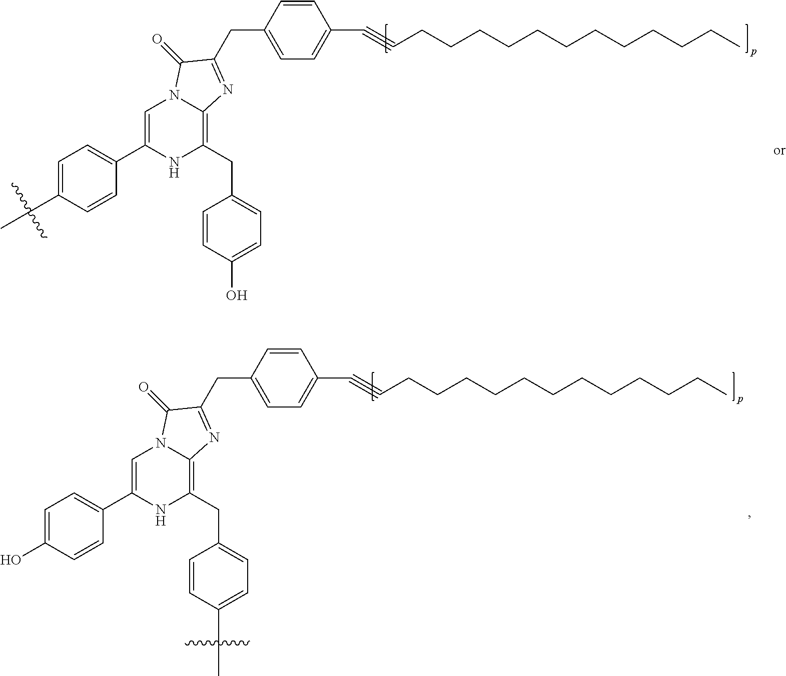

wherein p may be an integer ranging from 1 to 20. The structures herein are shown with attachment points to the linker Depending on the atom at the attachment point, upon cleavage of the linker to release Y, it is understood that the released moiety may include a leaving group X and/or X2 attached to any one of the structures shown herein.

In some embodiments, Y comprises an optionally substituted membrane-permeant coelenterazine moiety of the formula:

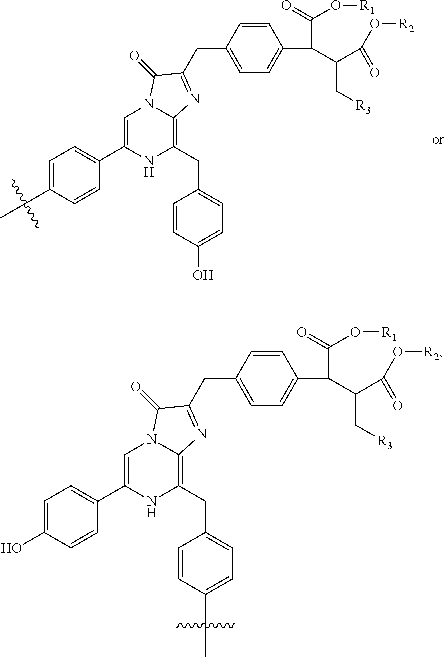

wherein R₁, R₂, and R₃ are independently alkyl, optionally substituted alkyl, alkenyl, or aralkyl. The structures herein are shown with attachment points to the linker Depending on the atom at the attachment point, upon cleavage of the linker to release Y, it is understood that the released moiety may include a leaving group X and/or X2 attached to any one of the structures shown herein.

In some embodiments, Y comprises an optionally substituted membrane-permeant coelenterazine moiety of the formula:

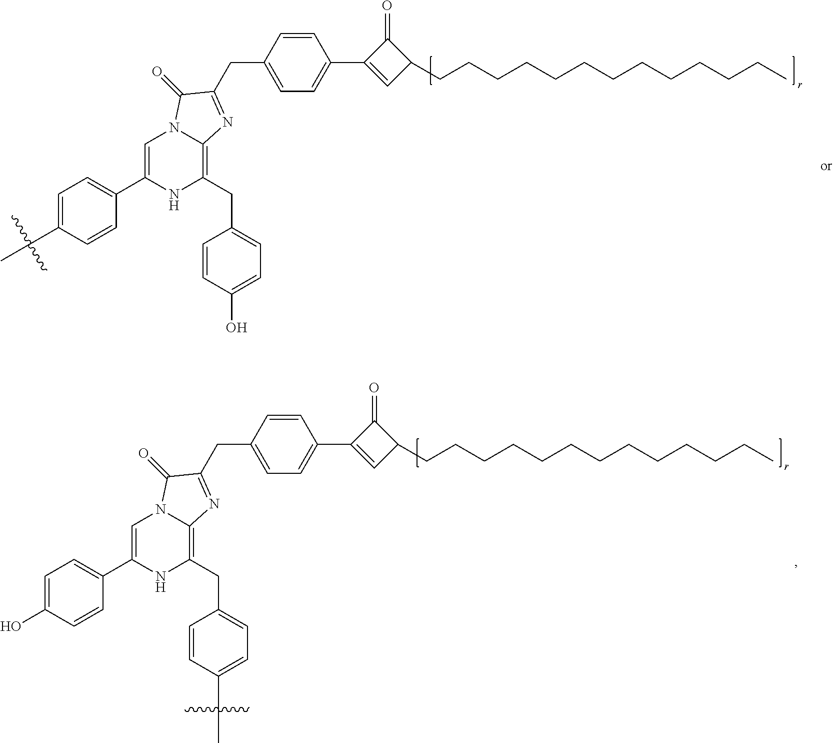

in which r may be an integer from 1 to 20. The structures herein are shown with attachment points to the linker Depending on the atom at the attachment point, upon cleavage of the linker to release Y, it is understood that the released moiety may include a leaving group X and/or X2 attached to any one of the structures shown herein.

In some embodiments, Y comprises an optionally substituted membrane-permeant coelenterazine moiety of the formula:

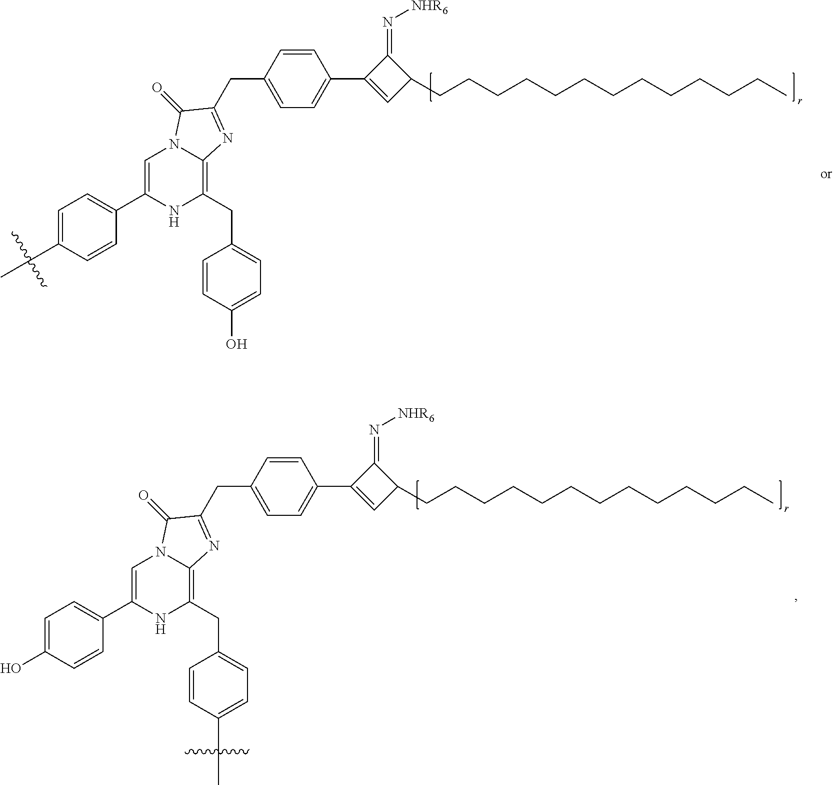

in which r may be an integer from 1 to 20 and R⁶ may be alkyl, aryl, aralkyl, optionally substituted alkyl, optionally substituted aryl, optionally substituted aralkyl, or alkoxyalkyl. The structures herein are shown with attachment points to the linker Depending on the atom at the attachment point, upon cleavage of the linker to release Y, it is understood that the released moiety may include a leaving group X and/or X2 attached to any one of the structures shown herein.

In some embodiments, Y comprises a moiety described by one of the following structures:

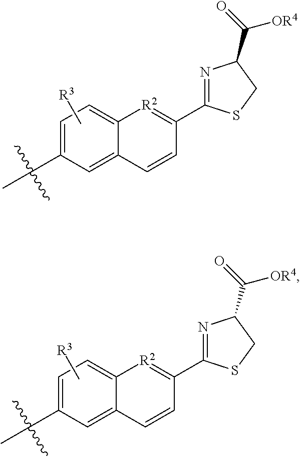

where R² is N or CH; R³ is hydrogen, halo, hydroxy, alkyl (e.g., methyl), alkoxy, amino, substituted amino (e.g., —NRR′), —CH₂N═R, or CH₂NRR′, where R and R′ are each independently selected from hydrogen, alkyl, aryl and heterocycle. The structures herein are shown with attachment points to the linker Depending on the atom at the attachment point, upon cleavage of the linker to release Y, it is understood that the released moiety may include a leaving group X and/or X2 attached to any one of the structures shown herein. In the above structure, the ring structures can be optionally substituted.

In some embodiments, Y comprises a moiety described by one of the following formulas:

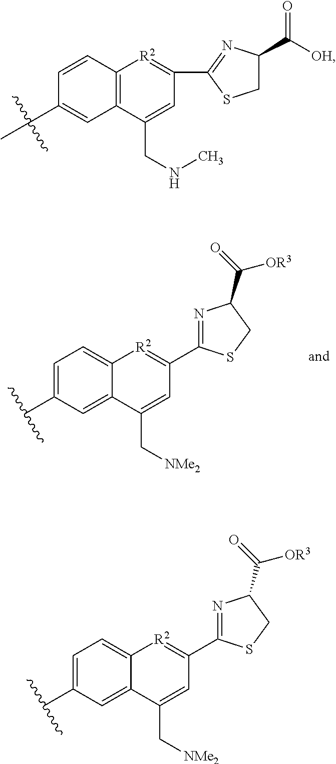

where R² is N or CH; and R³ is hydrogen, alkyl or substituted alkyl. The structures herein are shown with attachment points to the linker Depending on the atom at the attachment point, upon cleavage of the linker to release Y, it is understood that the released moiety may include a leaving group X and/or X2 attached to any one of the structures shown herein. In the above structure, the ring structures can be optionally substituted.

In some embodiments, Y comprises a moiety of one of the following formulas:

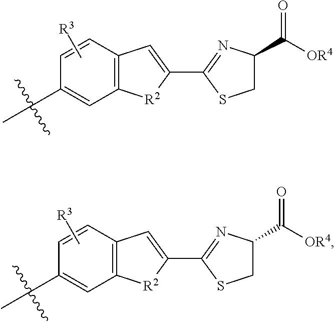

where R² is O or S; R³ is hydrogen, halo, hydroxyl, alkyl (e.g., methyl), alkoxy, amino, substituted amino (e.g., —NHRR′), —CH₂N═R, or CH₂NRR′, where R and R′ are each independently selected from hydrogen, alkyl, aryl and heterocycle; and R⁴ is hydrogen, alkyl or substituted alkyl. The structures herein are shown with attachment points to the linker Depending on the atom at the attachment point, upon cleavage of the linker to release Y, it is understood that the released moiety may include a leaving group X and/or X2 attached to any one of the structures shown herein. In the above structure, the ring structures can be optionally substituted.

In some embodiments, Y comprises a moiety of one of the following formulas

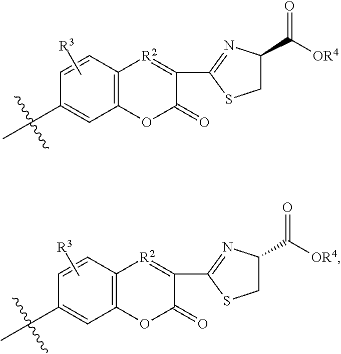

where R² is N or CH; R³ is hydrogen, halo, hydroxyl, alkyl (e.g., methyl), alkoxy, amino, substituted amino (e.g., —NHRR′), —CH₂N═R, or CH₂NRR′, wherein R and R′ are each independently selected from hydrogen, alkyl, aryl and heterocycle; and R⁴ is hydrogen, alkyl or substituted alkyl. The structures herein are shown with attachment points to the linker Depending on the atom at the attachment point, upon cleavage of the linker to release Y, it is understood that the released moiety may include a leaving group X and/or X2 attached to any one of the structures shown herein. In the above structure, the ring structures can be optionally substituted.

The disclosure provides a compound of formula (II):

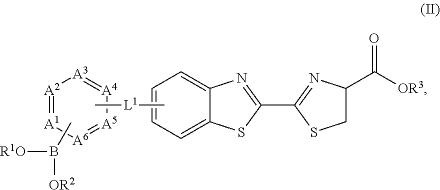

wherein
R¹ and R² are selected from hydrogen and alkyl; or and R² together form a boronic ester ring or substituted boronic ester ring;
each of A¹, A², A³, A⁴, A⁵, and A⁶ are independently selected from CH and N;
L¹ is cleavable linker group that provides for release of the benzothiazolyl core upon reaction of the —B(OR¹)(OR²) group with a reactive oxygen species; and
R³ is selected from hydrogen and alkyl.

In formula (II), R¹ and R² can be selected from hydrogen and alkyl; or R¹ and R² together can form a boronic ester ring or substituted boronic ester ring. In certain instances, both R¹ and R² are hydrogen. In certain instances, both R¹ and R² are alkyl, such as, for example, methyl, ethyl, propyl, isopropyl, and butyl. In certain instances, R¹ and R² together form a boronic ester ring or substituted boronic ester ring. In certain instances, R¹ and R² together form a boronic ester ring. In certain instances, R¹ and R² together form a substituted boronic ester ring. In certain instances, the —B(OR¹)(OR²) group is selected from the following:

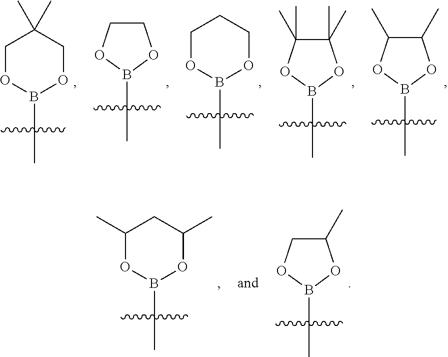

In formula (II), each of A¹, A², A³, A⁴, A⁵, and A⁶ can be independently selected from CH and N. In certain instances, all of A¹, A², A³, A⁴, A⁵, and A⁶ are CH. In certain instances, one of A¹, A², A³, A⁴, A⁵, and A⁶ is N and the rest are CH. In certain instances, two of A¹, A², A³, A⁴, A⁵, and A⁶ is N and the rest are CH.

In formula (II), L¹ is a cleavable linker group that provides for release of the benzothiazolyl core upon reaction of the —B(OR¹)(OR²) group with a reactive oxygen species (e.g., H₂O₂). Upon reaction of the —B(OR¹)(OR²) group with a reactive oxygen species, a cascade occurs in which an electron pair is donated into the linker. The L¹ linker group provides for release of the benzothiazolyl core by fragmentation or degradation of the linker with the donation of the electron pair, as described above.

The L¹ linker group can include one or more groups such as, but not limited to, alkyl, ether, carbamate, carbonate, carbamide (urea), ester, thioester, aryl, amide, imines, phosphate esters, hydrazones, acetals, orthoesters, and combinations thereof. In some embodiments, the L¹ linker group is described the following structure:

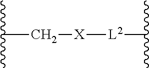

where X is a leaving group and L² is a linking group, wherein the bond that connects X to the adjacent —CH₂— group (e.g., CH₂—X) is a cleavable bond. In some embodiments X is oxygen or sulfur. In some embodiments, the leaving group is a carbamate, a carbonate, a thiol, an alcohol, an amino (e.g., an aryl amino) or a phenol group.

In certain embodiments, the linking group L² is a covalent bond or a chain of between 1 and 12 atoms in length (e.g., between 1 and 10, 1 and 8, 1 and 6 or 1 and 4 atoms in length). In some cases, L² is a chain of between 1 and 12 atoms in length that further includes a second leaving group adjacent to the detectable moiety Y (e.g., L² has a structure L³-X² where L³ is a linking group and X² is the second leaving group, e.g., O, NH or NR where R is an alkyl), such that upon cleavage of the cleavable bond (CH₂—X), a moiety is released (e.g., HX-L³-X²—Y) that includes both the first leaving group (X), L³-X² and Y. In such cases, the released moiety (e.g., HX-L³-X²—Y) may undergo further cleavage or fragmentation (e.g., via an intramolecular cyclization-release) to release HX²—Y, a moiety that may be directly or indirectly detected (e.g., a luciferin or aminoluciferin). In some embodiments, L² is a covalent bond, such that upon cleavage of the cleavable bond (CH₂—X), a moiety is released (e.g., HX—Y) that includes both the leaving group X and the detectable moiety Y, that together may be directly or indirectly detected. When referring to the detectable moiety that is released (e.g., a luciferin moiety) it is understood that the leaving group (X and/or X²) and segments of the linker may be attached to the detectable moiety being described. It is understood that in any of the embodiments described herein that upon cleavage of the cleavable bond of the linker, a moiety is released that may be directly or indirectly detected, or that may undergo further cleavage/fragmentation (e.g., via an intramolecular cyclization-release) prior to being directly or indirectly detected.

In certain instances, the L¹ linker group is selected from the following:

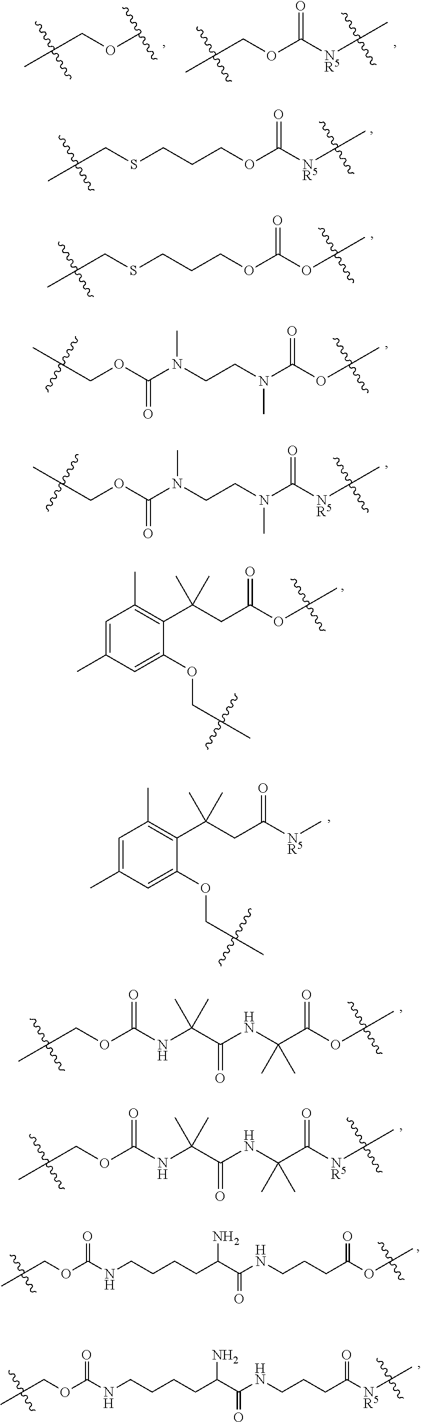

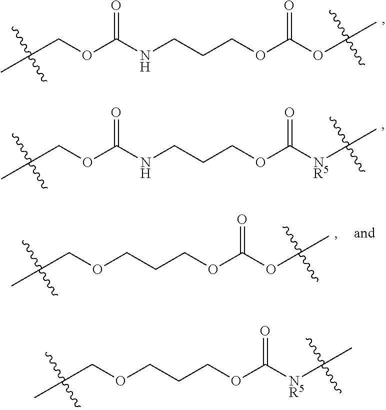

where R⁵ is hydrogen, alkyl, substituted alkyl or alkoxy, where optionally R⁵ may be covalently connected to Y (e.g., to form a fused ring system).

In formula (II), R³ is selected from hydrogen and alkyl. In certain instances, R³ is hydrogen. In certain instances, R³ is alkyl. In certain instances, R³ is methyl, ethyl, propyl, or butyl. In certain instances, R³ is methyl.

The disclosure provides a compound of formula (III):

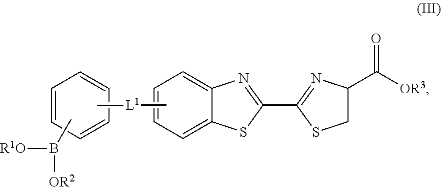

wherein

R¹ and R² are selected from hydrogen and alkyl; or R¹ and R² together form a boronic ester ring or substituted boronic ester ring;

L¹ is cleavable linker group that provides for release of the benzothiazolyl core upon reaction of the —B(OR¹)(OR²) group with a reactive oxygen species; and
R³ is selected from hydrogen and alkyl.

In formula (III), R¹ and R² can be selected from hydrogen and alkyl; or R¹ and R² together can form a boronic ester ring or substituted boronic ester ring. In certain instances, both R¹ and R² are hydrogen. In certain instances, both R¹ and R² are alkyl, such as, for example, methyl, ethyl, propyl, isopropyl, and butyl. In certain instances, R¹ and R² together form a boronic ester ring or substituted boronic ester ring. In certain instances, R¹ and R² together form a boronic ester ring. In certain instances, R¹ and R² together form a substituted boronic ester ring. In certain instances, the —B(OR¹)(OR²) group is selected from the following:

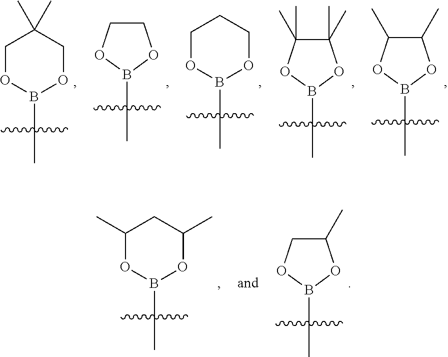

In formula (III), L¹ is cleavable linker group that provides for release of the benzothiazolyl core upon reaction of the —B(OR¹)(OR²) group with a reactive oxygen species. Upon reaction of the —B(OR¹)(OR²) group with a reactive oxygen species, a cascade occurs in which an electron pair is donated into the linker. The L¹ linker group provides for release of the benzothiazolyl core by fragmentation or cleavage of the linker with the donation of the electron pair, as described above.

The L¹ linker group can include one or more groups such as, but not limited to, alkyl, ether, carbamate, carbonate, carbamide (urea), ester, thioester, aryl, amide, imines, phosphate esters, hydrazones, acetals, orthoesters, and combinations thereof. In some embodiments, the L¹ linker group is described the following structure:

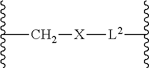

where X is a leaving group and L² is a linking group, wherein the bond that connects X to the adjacent —CH₂— group (e.g., CH₂—X) is a cleavable bond. In some embodiments X is oxygen or sulfur. In some embodiments, the leaving group is a carbamate, a carbonate, a thiol, an alcohol, an amino (e.g., an aryl amino) or a phenol group.

In certain embodiments, the linking group L² is a covalent bond or a chain of between 1 and 12 atoms in length (e.g., between 1 and 10, 1 and 8, 1 and 6 or 1 and 4 atoms in length). In some cases, L² is a chain of between 1 and 12 atoms in length that further includes a second leaving group adjacent to the detectable moiety Y (e.g., L² has a structure L³-X² where L³ is a linking group and X² is the second leaving group, e.g., O, NH or NR where R is an alkyl), such that upon cleavage of the cleavable bond (CH₂—X), a moiety is released (e.g., HX-L³-X²—Y) that includes both the first leaving group (X), L³-X² and Y. In such cases, the released moiety (e.g., HX-L³-X²—Y) may undergo further cleavage or fragmentation (e.g., via an intramolecular cyclization-release) to release HX²—Y, a moiety that may be directly or indirectly detected (e.g., a luciferin or aminoluciferin). In some embodiments, L² is a covalent bond, such that upon cleavage of the cleavable bond (CH₂—X), a moiety is released (e.g., HX—Y) that includes both the leaving group X and the detectable moiety Y, that together may be directly or indirectly detected. When referring to the detectable moiety that is released (e.g., a luciferin moiety) it is understood that the leaving group (X and/or X²) and segments of the linker may be attached to the detectable moiety being described. It is understood that in any of the embodiments described herein that upon cleavage of the cleavable bond of the linker, a moiety is released that may be directly or indirectly detected, or that may undergo further cleavage/fragmentation (e.g., via an intramolecular cyclization-release) prior to being directly or indirectly detected.

In certain instances, the L¹ linker group is selected from the following:

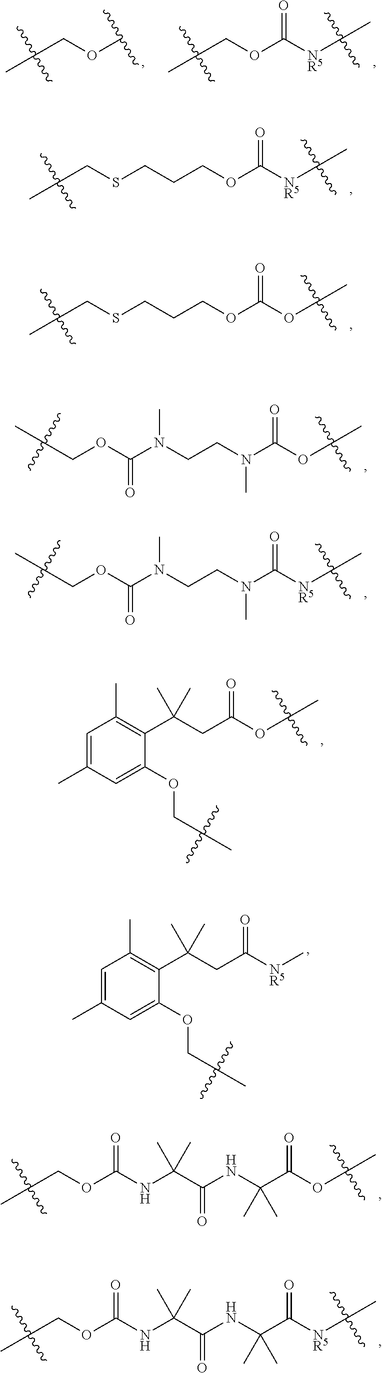

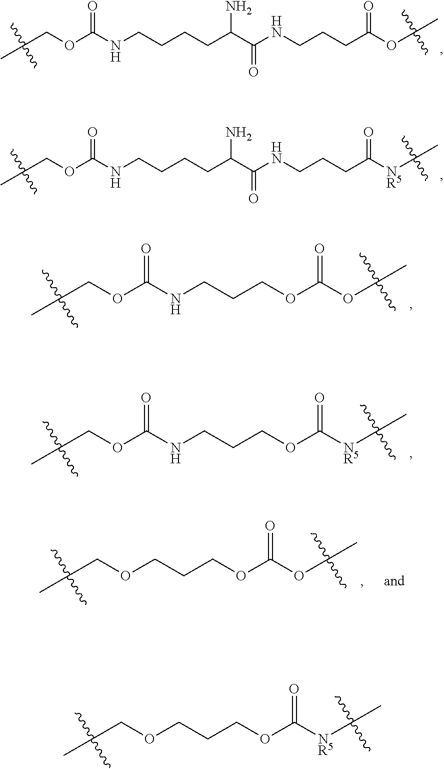

where R⁵ is hydrogen, alkyl, substituted alkyl or alkoxy, where optionally R⁵ may be covalently connected to Y (e.g., to form a fused ring system).

In formula (III), R³ is selected from hydrogen and alkyl. In certain instances, R³ is hydrogen. In certain instances, R³ is alkyl. In certain instances, R³ is methyl, ethyl, propyl, or butyl. In certain instances, R³ is methyl.

The disclosure provides a compound of formula (IV):

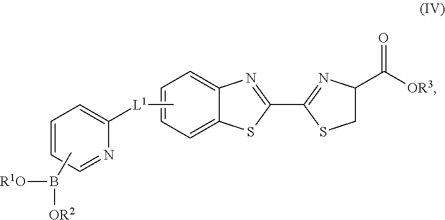

wherein
R¹ and R² are selected from hydrogen and alkyl; or R¹ and R² together form a boronic ester ring or substituted boronic ester ring;
L¹ is cleavable linker group that provides for release of the benzothiazolyl core upon reaction of the —B(OR¹)(OR²) group with a reactive oxygen species; and
R³ is selected from hydrogen and alkyl.

In formula (IV), R¹ and R² can be selected from hydrogen and alkyl; or R¹ and R² together can form a boronic ester ring or substituted boronic ester ring. In certain instances, both R¹ and R² are hydrogen. In certain instances, both R¹ and R² are alkyl, such as, for example, methyl, ethyl, propyl, isopropyl, and butyl. In certain instances, R¹ and R² together form a boronic ester ring or substituted boronic ester ring. In certain instances, R¹ and R² together form a boronic ester ring. In certain instances, R¹ and R² together form a substituted boronic ester ring. In certain instances, the —B(OR¹)(OR²) group is selected from the following:

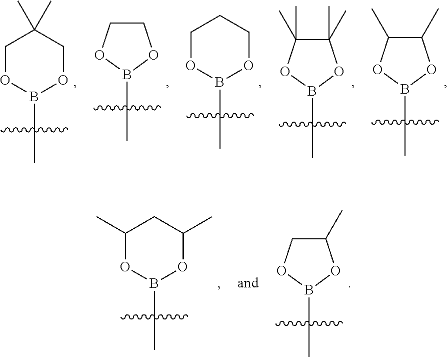

In formula (IV), L¹ is cleavable linker group that provides for release of the benzothiazolyl core upon reaction of the —B(OR¹)(OR²) group with a reactive oxygen species. Upon reaction of the —B(OR¹)(OR²) group with a reactive oxygen species, a cascade occurs in which an electron pair is donated into the linker. The L¹ linker group provides for release of the benzothiazolyl core by fragmentation or cleavage of the linker with the donation of the electron pair, as described above.

The L¹ linker group can include one or more groups such as, but not limited to, alkyl, ether, carbamate, carbonate, carbamide (urea), ester, thioester, aryl, amide, imines, phosphate esters, hydrazones, acetals, orthoesters, and combinations thereof. In some embodiments, the L¹ linker group is described the following structure:

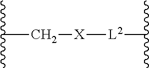

where X is a leaving group and L² is a linking group, wherein the bond that connects X to the adjacent —CH₂— group (e.g., CH₂—X) is a cleavable bond. In some embodiments X is oxygen or sulfur. In some embodiments, the leaving group is a carbamate, a carbonate, a thiol, an alcohol, an amino (e.g., an aryl amino) or a phenol group.

In certain embodiments, the linking group L² is a covalent bond or a chain of between 1 and 12 atoms in length (e.g., between 1 and 10, 1 and 8, 1 and 6 or 1 and 4 atoms in length). In some cases, L² is a chain of between 1 and 12 atoms in length that further includes a second leaving group adjacent to the detectable moiety Y (e.g., L² has a structure L³-X² where L³ is a linking group and X² is the second leaving group, e.g., O, NH or NR where R is an alkyl), such that upon cleavage of the cleavable bond (CH₂—X), a moiety is released (e.g., HX-L³-X²—Y) that includes both the first leaving group (X), L³-X² and Y. In such cases, the released moiety (e.g., HX-L³-X²—Y) may undergo further cleavage or fragmentation (e.g., via an intramolecular cyclization-release) to release HX²—Y, a moiety that may be directly or indirectly detected (e.g., a luciferin or aminoluciferin). In some embodiments, L² is a covalent bond, such that upon cleavage of the cleavable bond (CH₂—X), a moiety is released (e.g., HX—Y) that includes both the leaving group X and the detectable moiety Y, that together may be directly or indirectly detected. When referring to the detectable moiety that is released (e.g., a luciferin moiety) it is understood that the leaving group (X and/or X²) and segments of the linker may be attached to the detectable moiety being described. It is understood that in any of the embodiments described herein that upon cleavage of the cleavable bond of the linker, a moiety is released that may be directly or indirectly detected, or that may undergo further cleavage/fragmentation (e.g., via an intramolecular cyclization-release) prior to being directly or indirectly detected.

In certain instances, the L¹ linker group is selected from the following:

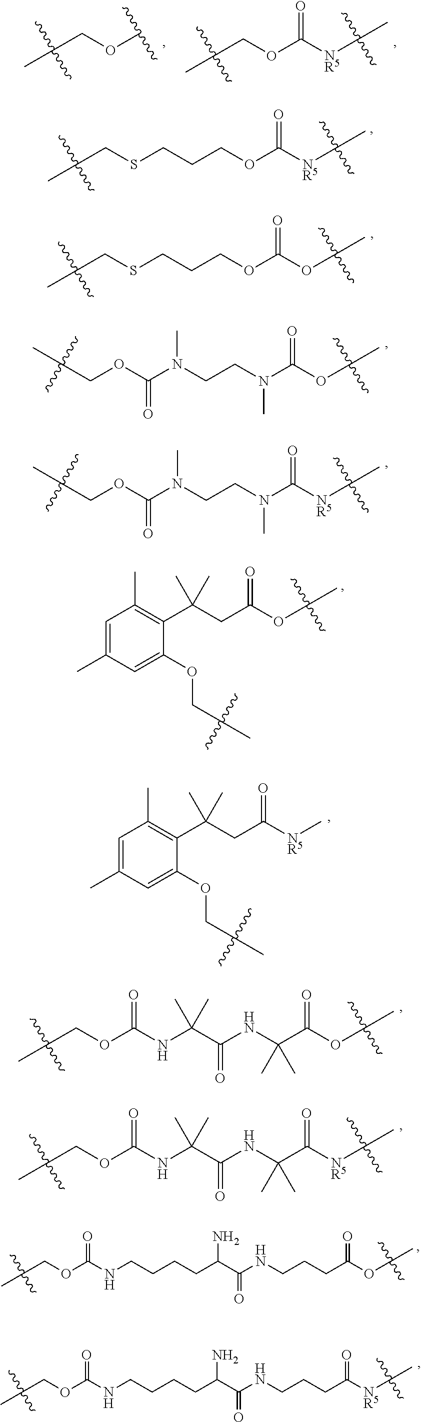

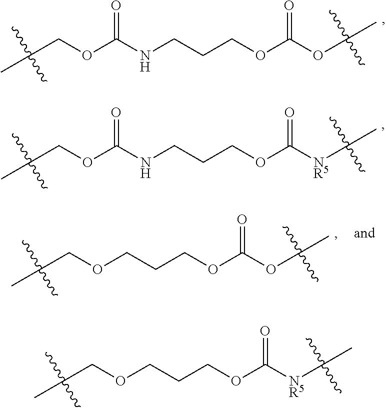

where R⁵ is hydrogen, alkyl, substituted alkyl or alkoxy, where optionally R⁵ may be covalently connected to Y (e.g., to form a fused ring system).

In formula (IV), R³ is selected from hydrogen and alkyl. In certain instances, R³ is hydrogen. In certain instances, R³ is alkyl. In certain instances, R³ is methyl, ethyl, propyl, or butyl. In certain instances, R³ is methyl.

The disclosure provides a compound of formula (V):

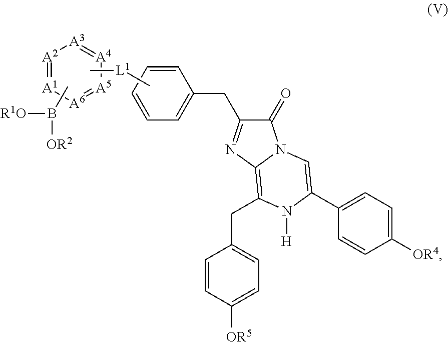

wherein
R¹ and R² are selected from hydrogen and alkyl; or and R² together form a boronic ester ring or substituted boronic ester ring;
each of A¹, A², A³, A⁴, A⁵, and A⁶ are independently selected from CH and N;
L¹ is cleavable linker group that provides for release of the phenyl core upon reaction of the —B(OR¹)(OR²) group with a reactive oxygen species;
R⁴ is selected from hydrogen and alkyl; and
R⁵ is selected from hydrogen and alkyl.

In formula (V), R¹ and R² can be selected from hydrogen and alkyl; or R¹ and R² together can form a boronic ester ring or substituted boronic ester ring. In certain instances, both R¹ and R² are hydrogen. In certain instances, both R¹ and R² are alkyl, such as, for example, methyl, ethyl, propyl, isopropyl, and butyl. In certain instances, R¹ and R² together form a boronic ester ring or substituted boronic ester ring. In certain instances, R¹ and R² together form a boronic ester ring. In certain instances, R¹ and R² together form a substituted boronic ester ring. In certain instances, the —B(OR¹)(OR²) group is selected from the following:

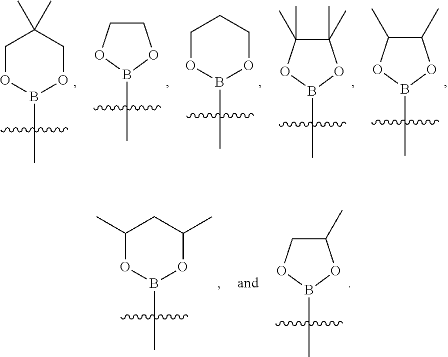

In formula (V), each of A¹, A², A³, A⁴, A⁵, and A⁶ can be independently selected from CH and N. In certain instances, all of A¹, A², A³, A⁴, A⁵, and A⁶ are CH. In certain instances, one of A¹, A², A³, A⁴, A⁵, and A⁶ is N and the rest are CH. In certain instances, two of A¹, A², A³, A⁴, A⁵, and A⁶ is N and the rest are CH.

In formula (V), L¹ is cleavable linker group that provides for release of the phenyl core upon reaction of the —B(OR¹)(OR²) group with a reactive oxygen species. Upon reaction of the —B(OR¹)(OR²) group with a reactive oxygen species, a cascade occurs in which an electron pair is donated into the linker. The L¹ linker group provides for release of the phenyl core by fragmentation or cleavage of the linker with the donation of the electron pair, as described above.

The L¹ linker group can include one or more groups such as, but not limited to, alkyl, ether, carbamate, carbonate, carbamide (urea), ester, thioester, aryl, amide, imines, phosphate esters, hydrazones, acetals, orthoesters, and combinations thereof. In some embodiments, the L¹ linker group is described the following structure:

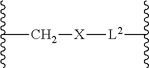

where X is a leaving group and L² is a linking group, wherein the bond that connects X to the adjacent —CH₂— group (e.g., CH₂—X) is a cleavable bond. In some embodiments X is oxygen or sulfur. In some embodiments, the leaving group is a carbamate, a carbonate, a thiol, an alcohol, an amino (e.g., an aryl amino) or a phenol group.

In certain embodiments, the linking group L² is a covalent bond or a chain of between 1 and 12 atoms in length (e.g., between 1 and 10, 1 and 8, 1 and 6 or 1 and 4 atoms in length). In some cases, L² is a chain of between 1 and 12 atoms in length that further includes a second leaving group adjacent to the detectable moiety Y (e.g., L² has a structure L³-X² where L³ is a linking group and X² is the second leaving group, e.g., O, NH or NR where R is an alkyl), such that upon cleavage of the cleavable bond (CH₂—X), a moiety is released (e.g., HX-L³-X²—Y) that includes both the first leaving group (X), L³-X² and Y. In such cases, the released moiety (e.g., HX-L³-X²—Y) may undergo further cleavage or fragmentation (e.g., via an intramolecular cyclization-release) to release HX²—Y, a moiety that may be directly or indirectly detected (e.g., a luciferin or aminoluciferin). In some embodiments, L² is a covalent bond, such that upon cleavage of the cleavable bond (CH₂—X), a moiety is released (e.g., HX—Y) that includes both the leaving group X and the detectable moiety Y, that together may be directly or indirectly detected. When referring to the detectable moiety that is released (e.g., a luciferin moiety) it is understood that the leaving group (X and/or X²) and segments of the linker may be attached to the detectable moiety being described. It is understood that in any of the embodiments described herein that upon cleavage of the cleavable bond of the linker, a moiety is released that may be directly or indirectly detected, or that may undergo further cleavage/fragmentation (e.g., via an intramolecular cyclization-release) prior to being directly or indirectly detected.

In certain instances, the L¹ linker group is selected from the following:

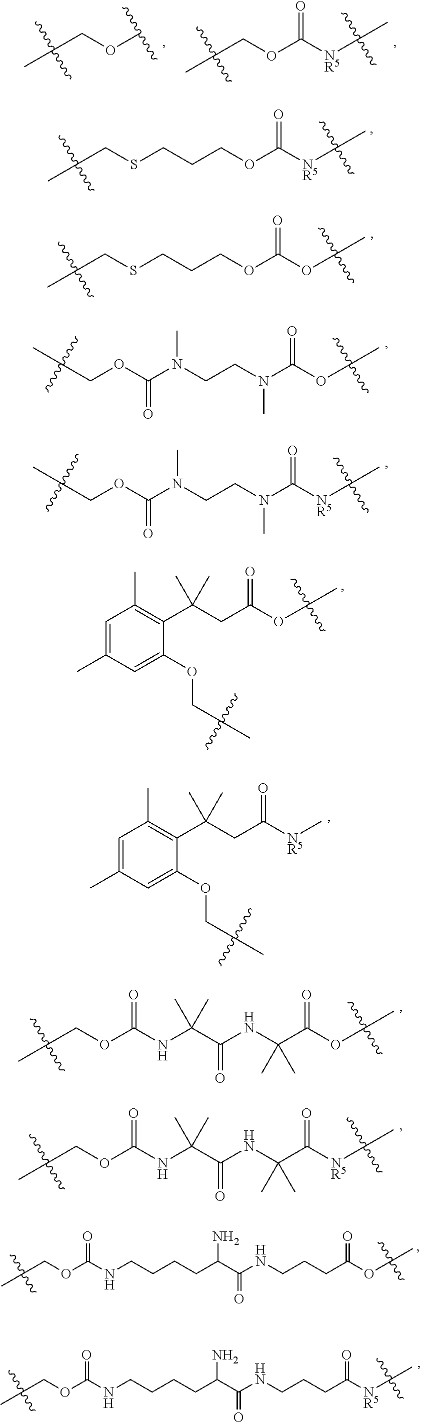

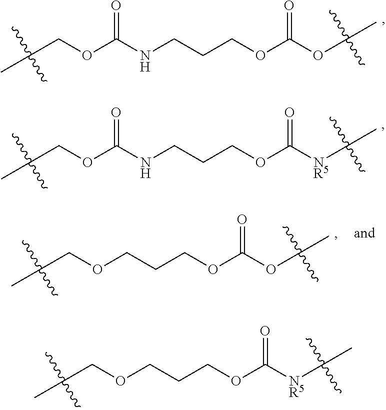

where R⁵ is hydrogen, alkyl, substituted alkyl or alkoxy, where optionally R⁵ may be covalently connected to Y (e.g., to form a fused ring system).

In formula (V), R⁴ is selected from hydrogen and alkyl. In certain instances, R⁴ is hydrogen. In certain instances, R⁴ is alkyl. In certain instances, R⁴ is methyl, ethyl, propyl, or butyl. In certain instances, R⁴ is methyl.

In formula (V), R⁵ is selected from hydrogen and alkyl. In certain instances, R⁵ is hydrogen. In certain instances, R⁵ is alkyl. In certain instances, R⁵ is methyl, ethyl, propyl, or butyl. In certain instances, R⁵ is methyl.

The disclosure provides a compound of formula (VI):

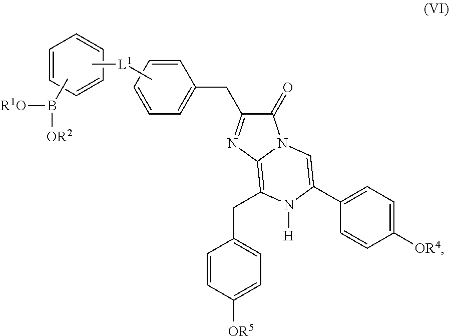

wherein
R¹ and R² are selected from hydrogen and alkyl; or R¹ and R² together form a boronic ester ring or substituted boronic ester ring;
L¹ is cleavable linker group that provides for release of the phenyl core upon reaction of the —B(OR¹)(OR²) group with a reactive oxygen species; and
R⁴ is selected from hydrogen and alkyl; and
R⁵ is selected from hydrogen and alkyl.

In formula (VI), R¹ and R² can be selected from hydrogen and alkyl; or R¹ and R² together can form a boronic ester ring or substituted boronic ester ring. In certain instances, both R¹ and R² are hydrogen. In certain instances, both R¹ and R² are alkyl, such as, for example, methyl, ethyl, propyl, isopropyl, and butyl. In certain instances, R¹ and R² together form a boronic ester ring or substituted boronic ester ring. In certain instances, R¹ and R² together form a boronic ester ring. In certain instances, R¹ and R² together form a substituted boronic ester ring. In certain instances, the —B(OR¹)(OR²) group is selected from the following:

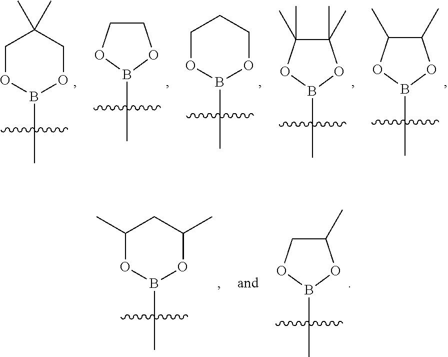

In formula (VI), L¹ is cleavable linker group that provides for release of the phenyl core upon reaction of the —B(OR¹)(OR²) group with a reactive oxygen species. Upon reaction of the —B(OR¹)(OR²) group with a reactive oxygen species, a cascade occurs in which an electron pair is donated into the linker. The L¹ linker group provides for release of the phenyl core by fragmentation or cleavage of the linker with the donation of the electron pair, as described above.

The L¹ linker group can include one or more groups such as, but not limited to, alkyl, ether, carbamate, carbonate, carbamide (urea), ester, thioester, aryl, amide, imines, phosphate esters, hydrazones, acetals, orthoesters, and combinations thereof. In some embodiments, the L¹ linker group is described the following structure:

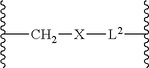

where X is a leaving group and L² is a linking group, wherein the bond that connects X to the adjacent —CH₂— group (e.g., CH₂—X) is a cleavable bond. In some embodiments X is oxygen or sulfur. In some embodiments, the leaving group is a carbamate, a carbonate, a thiol, an alcohol, an amino (e.g., an aryl amino) or a phenol group.

In certain embodiments, the linking group L² is a covalent bond or a chain of between 1 and 12 atoms in length (e.g., between 1 and 10, 1 and 8, 1 and 6 or 1 and 4 atoms in length). In some cases, L² is a chain of between 1 and 12 atoms in length that further includes a second leaving group adjacent to the detectable moiety Y (e.g., L² has a structure L³-X² where L³ is a linking group and X² is the second leaving group, e.g., O, NH or NR where R is an alkyl), such that upon cleavage of the cleavable bond (CH₂—X), a moiety is released (e.g., HX-L³-X²—Y) that includes both the first leaving group (X), L³-X² and Y. In such cases, the released moiety (e.g., HX-L³-X²—Y) may undergo further cleavage or fragmentation (e.g., via an intramolecular cyclization-release) to release HX²—Y, a moiety that may be directly or indirectly detected (e.g., a luciferin or aminoluciferin). In some embodiments, L² is a covalent bond, such that upon cleavage of the cleavable bond (CH₂—X), a moiety is released (e.g., HX—Y) that includes both the leaving group X and the detectable moiety Y, that together may be directly or indirectly detected. When referring to the detectable moiety that is released (e.g., a luciferin moiety) it is understood that the leaving group (X and/or X²) and segments of the linker may be attached to the detectable moiety being described. It is understood that in any of the embodiments described herein that upon cleavage of the cleavable bond of the linker, a moiety is released that may be directly or indirectly detected, or that may undergo further cleavage/fragmentation (e.g., via an intramolecular cyclization-release) prior to being directly or indirectly detected.

In certain instances, the L¹ linker group is selected from the following:

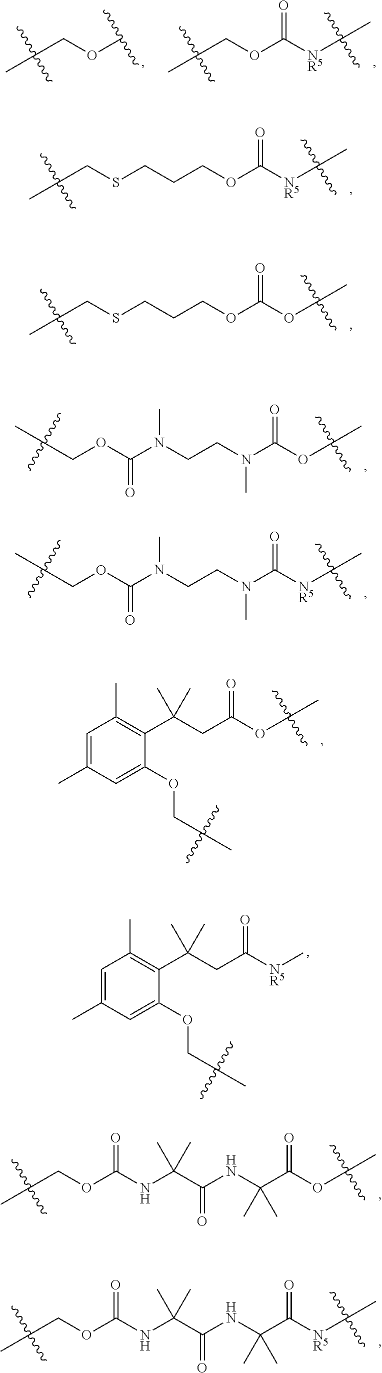

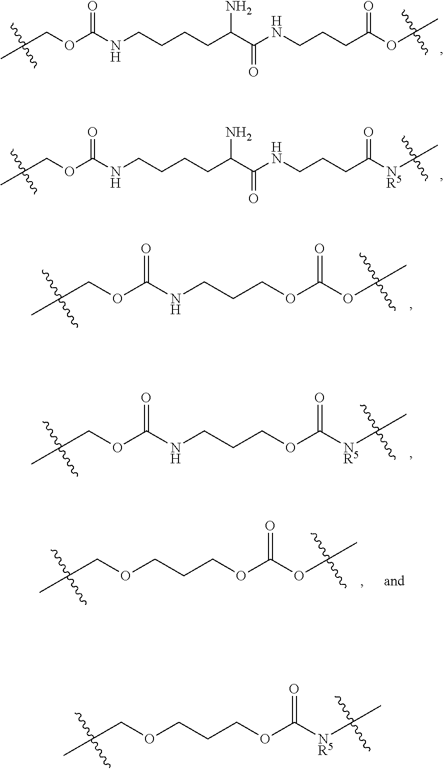

where R⁵ is hydrogen, alkyl, substituted alkyl or alkoxy, where optionally R⁵ may be covalently connected to Y (e.g., to form a fused ring system).

In formula (VI), R⁴ is selected from hydrogen and alkyl. In certain instances, R⁴ is hydrogen. In certain instances, R⁴ is alkyl. In certain instances, R⁴ is methyl, ethyl, propyl, or butyl. In certain instances, R⁴ is methyl.

In formula (VI), R⁵ is selected from hydrogen and alkyl. In certain instances, R⁵ is hydrogen. In certain instances, R⁵ is alkyl. In certain instances, R⁵ is methyl, ethyl, propyl, or butyl. In certain instances, R⁵ is methyl.

The disclosure provides a compound of formula (VII):

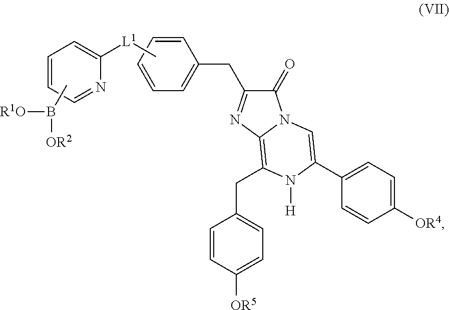

wherein
R¹ and R² are selected from hydrogen and alkyl; or R¹ and R² together form a boronic ester ring or substituted boronic ester ring;
L¹ is cleavable linker group that provides for release of the phenyl core upon reaction of the —B(OR¹)(OR²) group with a reactive oxygen species; and
R⁴ is selected from hydrogen and alkyl; and
R⁵ is selected from hydrogen and alkyl.

In formula (VII), R¹ and R² can be selected from hydrogen and alkyl; or R¹ and R² together can form a boronic ester ring or substituted boronic ester ring. In certain instances, both R¹ and R² are hydrogen. In certain instances, both R¹ and R² are alkyl, such as, for example, methyl, ethyl, propyl, isopropyl, and butyl. In certain instances, R¹ and R² together form a boronic ester ring or substituted boronic ester ring. In certain instances, R¹ and R² together form a boronic ester ring. In certain instances, R¹ and R² together form a substituted boronic ester ring. In certain instances, the —B(OR¹)(OR²) group is selected from the following:

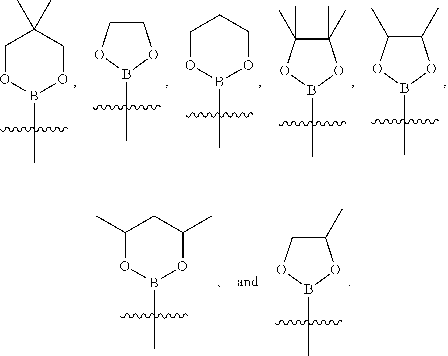

In formula (VII), L¹ is cleavable linker group that provides for release of the phenyl core upon reaction of the —B(OR¹)(OR²) group with a reactive oxygen species. Upon reaction of the —B(OR¹)(OR²) group with a reactive oxygen species, a cascade occurs in which an electron pair is donated into the linker. The L¹ linker group provides for release of the phenyl core by fragmentation or cleavage of the linker with the donation of the electron pair, as described above.

The L¹ linker group can include one or more groups such as, but not limited to, alkyl, ether, carbamate, carbonate, carbamide (urea), ester, thioester, aryl, amide, imines, phosphate esters, hydrazones, acetals, orthoesters, and combinations thereof. In some embodiments, the L¹ linker group is described the following structure:

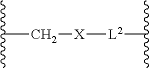

where X is a leaving group and L² is a linking group, wherein the bond that connects X to the adjacent —CH₂— group (e.g., CH₂—X) is a cleavable bond. In some embodiments X is oxygen or sulfur. In some embodiments, the leaving group is a carbamate, a carbonate, a thiol, an alcohol, an amino (e.g., an aryl amino) or a phenol group.

In certain embodiments, the linking group L² is a covalent bond or a chain of between 1 and 12 atoms in length (e.g., between 1 and 10, 1 and 8, 1 and 6 or 1 and 4 atoms in length). In some cases, L² is a chain of between 1 and 12 atoms in length that further includes a second leaving group adjacent to the detectable moiety Y (e.g., L² has a structure L³-X² where L³ is a linking group and X² is the second leaving group, e.g., O, NH or NR where R is an alkyl), such that upon cleavage of the cleavable bond (CH₂—X), a moiety is released (e.g., HX-L³-X²—Y) that includes both the first leaving group (X), L³-X² and Y. In such cases, the released moiety (e.g., HX-L³-X²—Y) may undergo further cleavage or fragmentation (e.g., via an intramolecular cyclization-release) to release HX²—Y, a moiety that may be directly or indirectly detected (e.g., a luciferin or aminoluciferin). In some embodiments, L² is a covalent bond, such that upon cleavage of the cleavable bond (CH₂—X), a moiety is released (e.g., HX—Y) that includes both the leaving group X and the detectable moiety Y, that together may be directly or indirectly detected. When referring to the detectable moiety that is released (e.g., a luciferin moiety) it is understood that the leaving group (X and/or X²) and segments of the linker may be attached to the detectable moiety being described. It is understood that in any of the embodiments described herein that upon cleavage of the cleavable bond of the linker, a moiety is released that may be directly or indirectly detected, or that may undergo further cleavage/fragmentation (e.g., via an intramolecular cyclization-release) prior to being directly or indirectly detected.

In certain instances, the L¹ linker group is selected from the following:

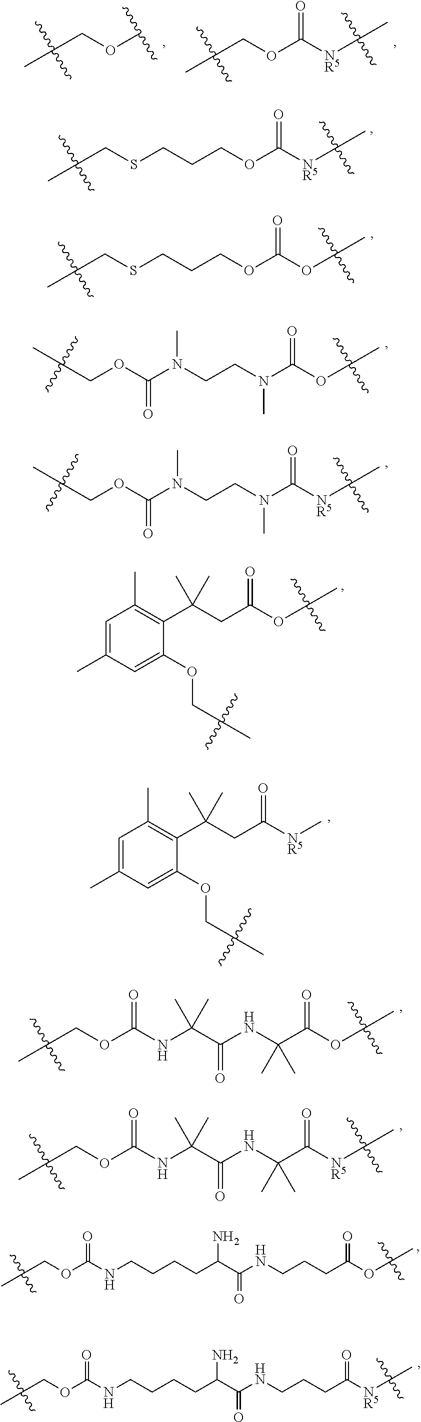

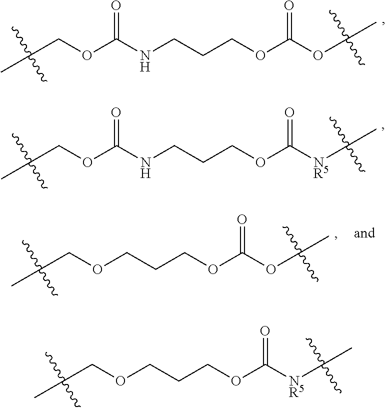

where R⁵ is hydrogen, alkyl, substituted alkyl or alkoxy, where optionally R⁵ may be covalently connected to Y (e.g., to form a fused ring system).

In formula (VII), R⁴ is selected from hydrogen and alkyl. In certain instances, R⁴ is hydrogen. In certain instances, R⁴ is alkyl. In certain instances, R⁴ is methyl, ethyl, propyl, or butyl. In certain instances, R⁴ is methyl.

In formula (VII), R⁵ is selected from hydrogen and alkyl. In certain instances, R⁵ is hydrogen. In certain instances, R⁵ is alkyl. In certain instances, R⁵ is methyl, ethyl, propyl, or butyl. In certain instances, R⁵ is methyl.

The following table shows representative compounds.

TABLE

| | —B(OR¹)(OR²)—A ring | L¹ | Y |
|---|---|---|---|
| 1 | 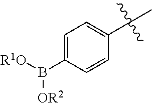 | 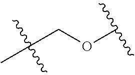 [SMILES: *CO*] | 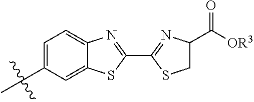 |
| 2 | 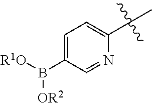 | 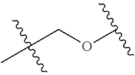 [SMILES: *CO*] | 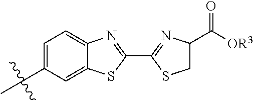 |
| 3 | 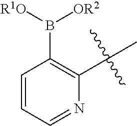 | 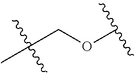 [SMILES: *CO*] | 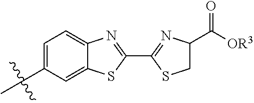 |
| 4 | 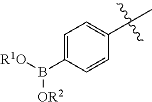 | 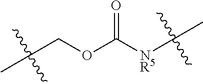 | 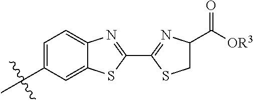 |
| 5 | 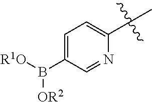 | 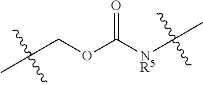 | 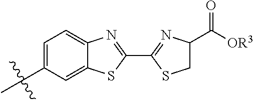 |
| 6 | 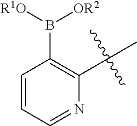 | 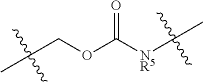 | 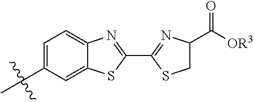 |
| 7 | 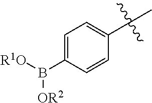 | 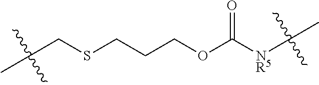 | 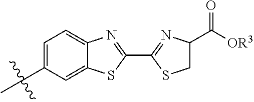 |
| 8 | 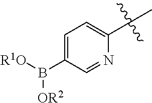 | 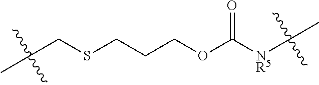 | 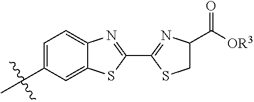 |
| 9 | 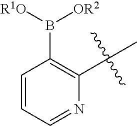 | 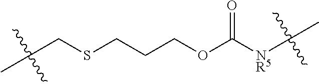 | 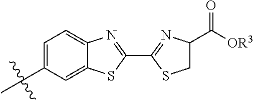 |
| 10 | 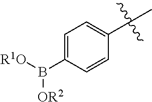 | 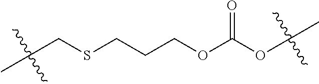 [SMILES: *CSCCCOC(=O)O*] | 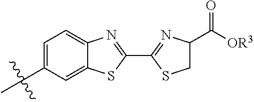 |
| 11 | 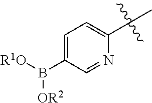 | 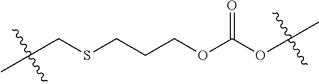 [SMILES: *CSCCCOC(=O)O*] | 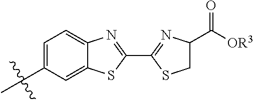 |
| 12 | 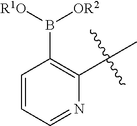 | 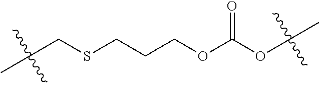 [SMILES: *CSCCCOC(=O)O*] | 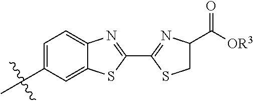 |
| 13 | 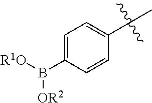 | 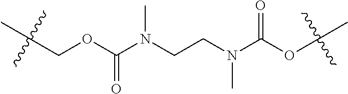 [SMILES: *COC(=O)N(C)CCN(C)C(=O)O*] | 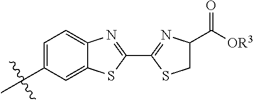 |
| 14 | 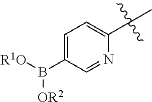 | 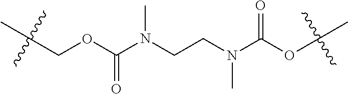 [SMILES: *COC(=O)N(C)CCN(C)C(=O)O*] | 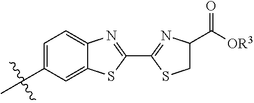 |
| 15 | 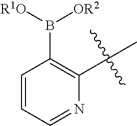 | 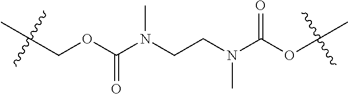 [SMILES: *COC(=O)N(C)CCN(C)C(=O)O*] | 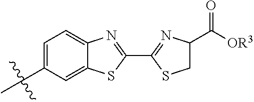 |
| 16 | 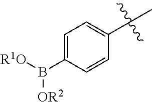 | 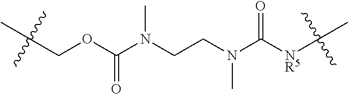 | 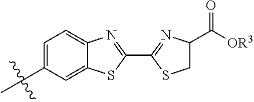 |
| 17 | 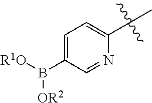 | 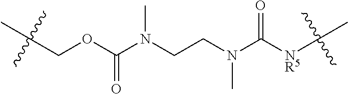 | 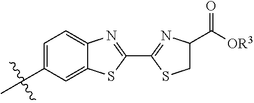 |
| 18 | 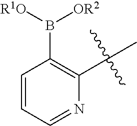 | 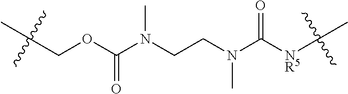 | 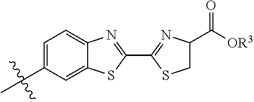 |
| 19 | 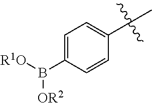 | 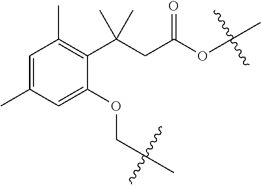 [SMILES: *COc1cc(C)cc(C)c1C(C)(C)CC(=O)O*] | 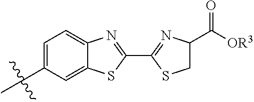 |
| 20 | 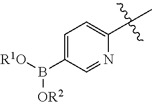 | 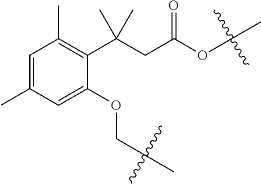 [SMILES: *COc1cc(C)cc(C)c1C(C)(C)CC(=O)O*] | 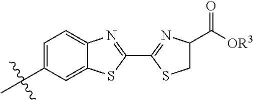 |
| 21 | 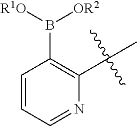 | 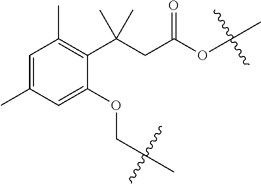 [SMILES: *COc1cc(C)cc(C)c1C(C)(C)CC(=O)O*] | 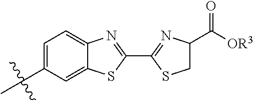 |
| 22 | 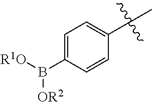 | 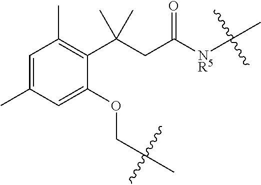 | 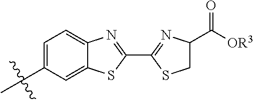 |
| 23 | 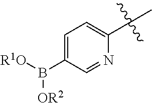 | 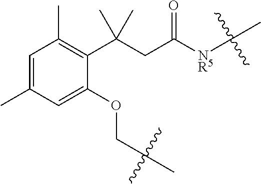 | 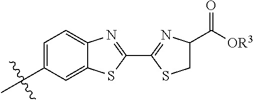 |
| 24 | 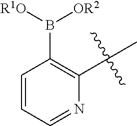 | 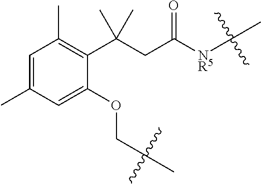 | 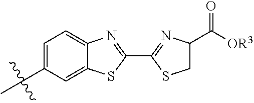 |
| 25 | 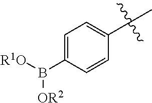 | 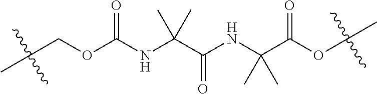 | 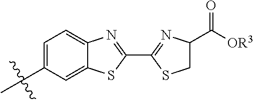 |
| 26 | 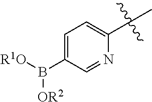 | 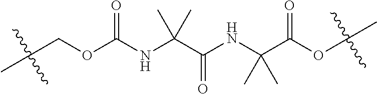 | 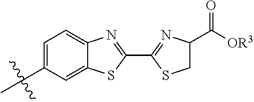 |
| 27 | 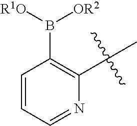 | 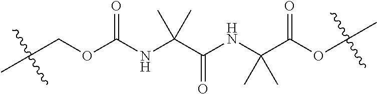 | 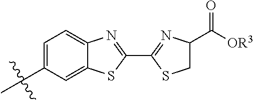 |
| 28 | 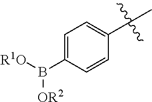 | 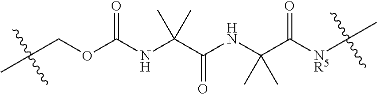 | 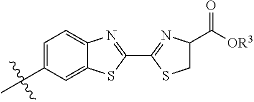 |
| 29 | 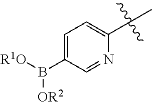 | 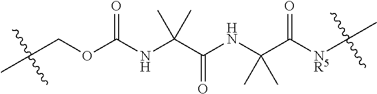 | 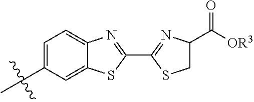 |
| 30 | 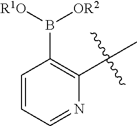 | 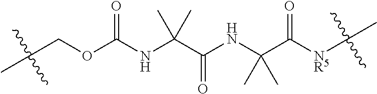 | 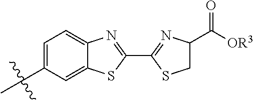 |
| 31 | 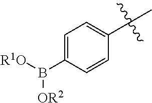 | 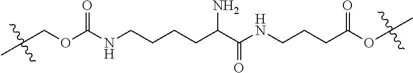 [SMILES: *COC(=O)NCCCCC(N)C(=O)NCCCC(=O)O*] | 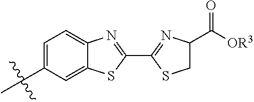 |
| 32 | 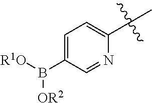 | 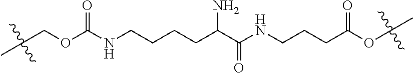 [SMILES: *COC(=O)NCCCCC(N)C(=O)NCCCC(=O)O*] | 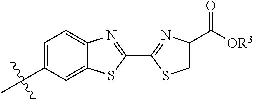 |
| 33 | 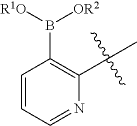 | 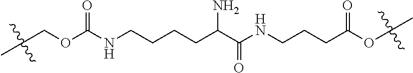 [SMILES: *COC(=O)NCCCCC(N)C(=O)NCCCC(=O)O*] | 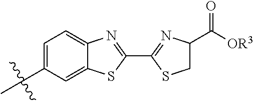 |
| 34 | 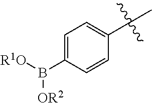 | 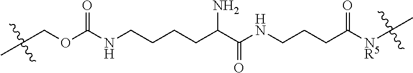 | 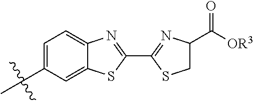 |
| 35 | 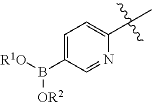 | 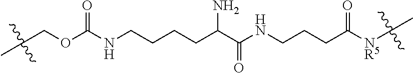 | 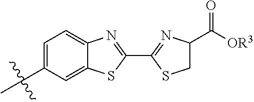 |
| 36 | 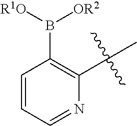 | 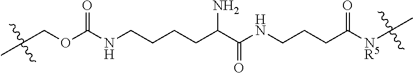 | 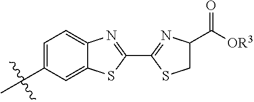 |
| 37 | 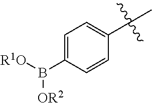 | 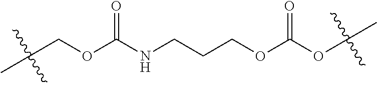 [SMILES: *COC(=O)NCCCOC(=O)O*] | 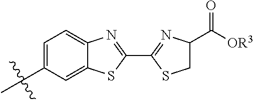 |
| 38 | 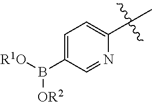 | 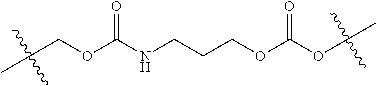 [SMILES: *COC(=O)NCCCOC(=O)O*] | 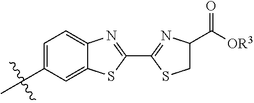 |
| 39 | 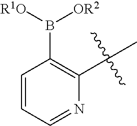 | 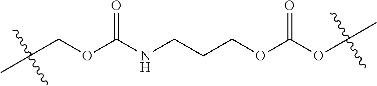 [SMILES: *COC(=O)NCCCOC(=O)O*] | 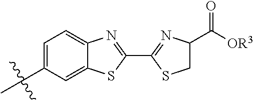 |
| 40 | 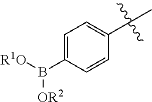 | 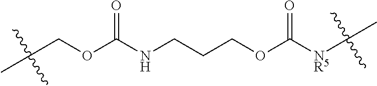 | 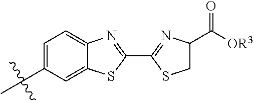 |
| 41 | 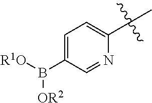 | 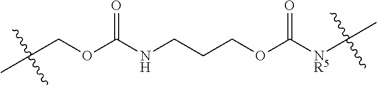 | 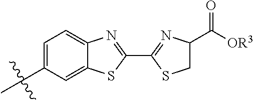 |
| 42 | 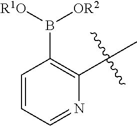 | 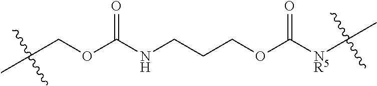 | 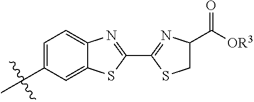 |
| 43 | 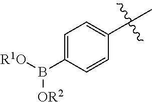 | 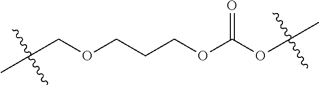 [SMILES: *COCCCOC(=O)O*] | 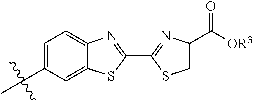 |
| 44 | 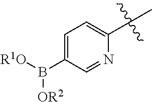 | 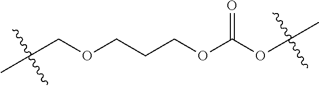 [SMILES: *COCCCOC(=O)O*] | 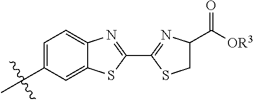 |
| 45 | 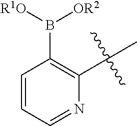 | 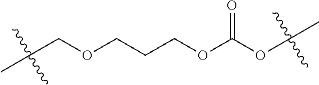 [SMILES: *COCCCOC(=O)O*] | 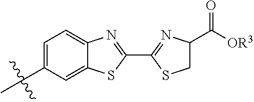 |
| 46 | 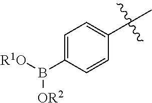 | 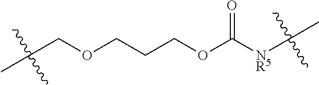 | 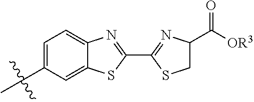 |
| 47 | 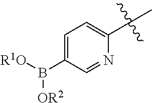 | 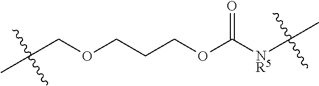 | 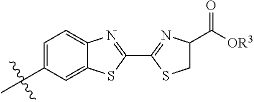 |
| 48 | 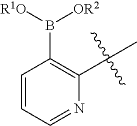 | 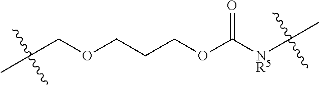 | 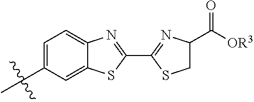 |

The present disclosure provides a compound of formula (VIII), where Formula VIII is Z-L¹-Y wherein:

### Z is (R¹O)(R²O)B—,

R¹ and R² are selected from hydrogen and alkyl; or R¹ and R² together form a boronic ester ring or substituted boronic ester ring;
L¹ is an optional cleavable linker group that provides for release of Y upon reaction of the —B(OR¹)(OR²) group with a reactive oxygen species; and
Y is a detectable moiety that is released upon reaction of the compound with a reactive oxygen species; wherein, after release, the detectable moiety generates a detectable signal, either directly (e.g., by fluorescence or luminescence) or indirectly (e.g., after an enzyme mediated reaction).

In Formula VIII, where L¹ is absent, Y is directly linked to Z.

The disclosure provides an optionally substituted coelenterazine derivative, formulae (IX), (X), or (XI):

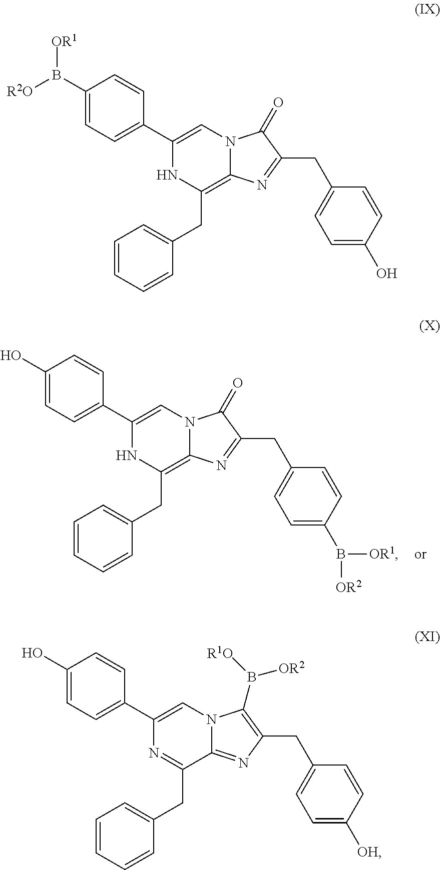

wherein R¹ and R² are selected from hydrogen and alkyl; or R¹ and R² together form a boronic ester ring or substituted boronic ester ring.

### Compositions

The present disclosure provides compositions, including pharmaceutical compositions, comprising a subject compound. Compositions comprising a subject compound can include one or more of: a salt, e.g., NaCl, MgCl, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a membrane penetration facilitator; and the like.

The present disclosure provides pharmaceutical compositions comprising a subject compound. A subject compound can be formulated with one or more pharmaceutically acceptable excipients. A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

A subject compound can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. In some cases, a suitable excipient is dimethylsulfoxide (DMSO). In other cases, DMSO is specifically excluded.

For oral preparations, a subject compound can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

A subject compound can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

A subject compound can be utilized in aerosol formulation to be administered via inhalation. A subject compound can be formulated into pressurized acceptable propellants such as dichlorodifluoromethane, propane, nitrogen and the like.

Furthermore, a subject compound can be made into suppositories by mixing with a variety of bases such as emulsifying bases or water-soluble bases. A subject compound can be administered rectally via a suppository. The suppository can include vehicles such as cocoa butter, carbowaxes and polyethylene glycol monomethyl ethers, which melt at body temperature, yet are solidified at room temperature.

### Utility

A subject compound, and a subject composition, finds use in various applications. A subject compound can be used in various diagnostic and detection methods.

### Detection of an ROS in a Living Cell In Vitro

The present disclosure provides a method of detecting an ROS (e.g., H₂O₂) in a living cell in vitro. In some embodiments, a subject detection method involves contacting a subject compound with a living cell in vitro, e.g., a subject compound is contacted with cells growing in suspension (e.g., as unicellular entities) or as a monolayer in in vitro cell culture; and detecting a signal generated by reaction of the compound with an ROS in the cell. The cells can be primary cells, non-transformed cells, cells isolated from an individual, immortalized cell lines, transformed cells, etc.

Non-limiting examples of cells are cells of multicellular organisms, e.g., cells of invertebrates and vertebrates, such as myoblasts, neutrophils, erythrocytes, osteoblasts, chondrocytes, basophils, eosinophils, adipocytes, invertebrate neurons (e.g., Helix aspera), vertebrate neurons, mammalian neurons, adrenomedullary cells, melanocytes, epithelial cells, and endothelial cells; tumor cells of all types (e.g., melanoma, myeloid leukemia, carcinomas of the lung, breast, ovaries, colon, kidney, prostate, pancreas and testes); cardiomyocytes, endothelial cells, lymphocytes (T-cell and B cell), mast cells, vascular intimal cells, hepatocytes, leukocytes including mononuclear leukocytes; stem cells such as hematopoietic stem cells, neural, skin, lung, kidney, liver and myocyte stem cells; osteoclasts, connective tissue cells, keratinocytes, melanocytes, hepatocytes, and kidney cells.

Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLL3), human embryonic kidney (HEK) cells (ATCC No. CRL1573), HLHepG2 cells, and the like.

In some cases, as discussed below, the cells in which the ROS (e.g., H₂O₂) is being detected are genetically modified to produce luciferase.

An ROS that is present in the extracellular medium of a living cell in vitro can also be detected by a subject method. In these cases, a subject detection method involves contacting a subject compound with a living cell in vitro, e.g., a subject compound is contacted with cells growing in suspension (e.g., as unicellular entities) or as a monolayer in in vitro cell culture; and detecting a signal generated by reaction of the compound with an ROS in the cell.

Suitable methods of detecting a signal generated by reaction of a subject compound with an ROS (e.g., H₂O₂) in a living cell in vitro include, e.g., microscopy, fluorescence activated cell sorting, spectroscopy (e.g., a multi-well plate reader that detects luminescence), luminometers, photomultiplier tubes, and the like.

### Detection of an ROS In Vivo, in a Living Cell In Vivo or in an Extracellular Compartment of a Multicellular Organism

The present disclosure provides a method of detecting an ROS (e.g., H₂O₂) in a living cell in vivo, e.g., in a living multicellular organism. In some embodiments, the method involves administering a subject compound (or a composition comprising a subject compound) to a multicellular organism (e.g., an individual such as a mammal); and detecting a signal generated by reaction of the compound with an ROS (e.g., H₂O₂) in a cell of the multicellular organism (e.g., in a cell of the individual). A subject detection method can also be carried out ex vivo, e.g., where a tissue or cells are taken from an individual and imaged.

The present disclosure also provides a method of detecting an ROS (e.g., H₂O₂) in a multicellular organism, where the ROS is present extracellularly in the multicellular organism. In some embodiments, the method involves administering a subject compound (or a composition comprising a subject compound) to a multicellular organism (e.g., an individual such as a mammal); and detecting a signal generated by reaction of the compound with an ROS (e.g., H₂O₂) in the multicellular organism, where the ROS is present extracellularly in the multicellular organism. The ROS can be present in an extracellular fluid (e.g., cerebrospinal fluid, lymph, plasma, and the like) or other extracellular environment.

Suitable methods of detecting a signal generated by reaction of a subject compound with an ROS (e.g., H₂O₂) in a living cell in vitro include, e.g., microscopy, fluorescence activated cell sorting, spectroscopy (e.g., a multi-well plate reader that detects luminescence), luminometers, photomultiplier tubes, and the like. Suitable methods of detecting a signal generated by reaction of a subject compound with an ROS (e.g., H₂O₂) in a living cell in vivo include, e.g., use of a charged-coupled device (CCD) camera; a cooled CCD camera; or any other device capable of bioluminescent imaging. Use of a CCD camera can allow three-dimensional imaging of the level of ROS.

In some of the above-discussed in vitro or in vivo embodiments, the cells in which the ROS (e.g., H₂O₂) is being detected are genetically modified to produce luciferase. Luciferase-encoding nucleic acids from any of a wide variety of vastly different species, e.g., the luciferase genes of Photinus pyralis and Photuris pennsylvanica (fireflies of North America), Pyrophorus plagiophthalamus (the Jamaican click beetle), Renilla reniformis (the sea pansy), and several bacteria (e.g., Xenorhabdus luminescens and Vibrio spp), can be used. In addition, variant luciferase can be used; see, e.g., variant luciferase described in U.S. Pat. No. 7,507,565. Numerous luciferase amino acid sequences (and corresponding encoding nucleotide sequences) are available; see, e.g., GenBank Accession Nos.: 1) BAH86766, and GenBank AB508949 for the corresponding encoding nucleotide sequence; 2) CAA59282 (Photinus pyralis) and GenBank X84847 for the corresponding encoding nucleotide sequence; 3) ABD66580.1 (Diaphenes pectinealis); 4) AAV32457.1 Cratomorphus distinctus); 5) AAR20792.1 (Pyrocoelia rufa); 6) AAR20794.1 (Lampyris notiluca); 7) AAL40677 (Pyrocystis lunula), and GenBank AF394059 for the corresponding encoding nucleotide sequence; and 8) AAV35380 (Pyrocystis noctiluca), and GenBank AY766385 for the corresponding encoding nucleotide sequence.

In some embodiments, the luciferase is encoded by a nucleotide sequence encoding the luciferase, and the nucleotide sequence is operably linked to a control element. Suitable control elements include promoters, enhancers, and the like. In some embodiments, the promoter is a constitutive promoter. In other embodiments, the promoter is an inducible promoter. In other embodiments, the promoter is a cell type-specific promoter. Such promoters are well known in the art.

In some embodiments, luciferase is expressed as a transgene in a non-human transgenic animal. In some embodiments, the luciferase is expressed in all cells of the transgenic non-human animal. In other embodiments, the luciferase is expressed in a subset of cells in the transgenic non-human animal. For example, in some embodiments, the luciferase is expressed only in neurons in the transgenic non-human animal. In these embodiments, the luciferase-encoding transgene comprises a nucleotide sequence encoding luciferase, where the nucleotide sequence is operably linked to a cell type-specific control element.

A subject detection method can be used to detect the level of an ROS (e.g., H₂O₂) in a cell in response to an internal or an external stimulus. External and internal signals (stimuli) include, but are not limited to, infection of a cell by a microorganism, including, but not limited to, a bacterium (e.g., Mycobacterium spp., Shigella, Chlamydia, and the like), a protozoan (e.g., Trypanosoma spp., Plasmodium spp., Toxoplasma spp., and the like), a fungus, a yeast (e.g., Candida spp.), or a virus (including viruses that infect mammalian cells, such as human immunodeficiency virus, foot and mouth disease virus, Epstein-Barr virus, and the like; viruses that infect plant cells; etc.); change in pH of the medium in which a cell is maintained or a change in internal pH; excessive heat relative to the normal range for the cell or the multicellular organism; excessive cold relative to the normal range for the cell or the multicellular organism; an effector molecule such as a hormone, a cytokine, a chemokine, a neurotransmitter; an ingested or applied drug; a ligand for a cell-surface receptor; a ligand for a receptor that exists internally in a cell, e.g., a nuclear receptor; hypoxia; a change in cyoskeleton structure; light; dark; caloric restriction; caloric intake; mitogens, including, but not limited to, lipopolysaccharide (LPS), pokeweed mitogen; stress; antigens; sleep pattern (e.g., sleep deprivation, alteration in sleep pattern, and the like); an apoptosis-inducing signal; electrical charge (e.g., a voltage signal); ion concentration of the medium in which a cell is maintained, or an internal ion concentration, exemplary ions including sodium ions, potassium ions, chloride ions, calcium ions, and the like; presence or absence of a nutrient; metal ions; a transcription factor; a tumor suppressor; cell-cell contact; adhesion to a surface; peptide aptamers; RNA aptamers; intrabodies; and the like.

For example, in some embodiments, a cell is contacted with a subject compound and an internal or external stimulus is applied; and the signal produced by the compound is detected and compared to the signal detected in the absence of the internal or external stimulus.

A subject detection method can be used to detect the level of an ROS (e.g., H₂O₂) in a cell (in vitro, ex vivo, or in vivo) as a function of a particular physiological state. For example, the level of an ROS (e.g., H₂O₂) is measured in a cell when the cell (e.g., a single cell in vitro; or a cell in a multicellular organism; or in an extracellular fluid in a multicellular organism) is in a first physiological state; and the level of the ROS (e.g., H₂O₂) is measured in the same cell when the cell is in a second physiological state. For example, the first physiological state could be the absence of disease or absence of a condition; and the second physiological state could be a disease state or a particular condition. Thus, for example, the level of an ROS (e.g., H₂O₂) can be measured in cells or tissues of individual to detect the presence of a disease state or a condition. Disease states and other conditions that are associated with altered ROS levels include, but are not limited to, cancer, inflammation, aging, cardiovascular disease, diabetes, neurodegenerative disease, and stroke. ROS levels in different cells in different physiological states, or in different organisms in different physiological states, can also be compared.

A subject detection method can be used to detect the level of an ROS (e.g., H₂O₂) in a cell (e.g., a single cell in vitro; or a cell in a multicellular organism; or in an extracellular fluid in a multicellular organism) over time. For example, the level of an ROS (e.g., H₂O₂) is detected at a first time and at a second time; and the levels of the ROS (e.g., H₂O₂) detected at the first and second times are compared. In some embodiments, the first time is before treatment with an agent (e.g., a therapeutic agent); and the second time is after treatment with an agent. In these embodiments, the level of ROS (e.g., H₂O₂) can be used to determine the effect of treatment of an individual with the agent. In other embodiments, the first time is at a first age of a multicellular organism; and the second time is at a second age of the multicellular organism. In these embodiments, the change in level of ROS (e.g., H₂O₂) with age can be monitored.

A subject compound can be used to determine the effect that an agent has on the level of an ROS (e.g. H₂O₂) in a cell and/or cells (e.g., a single cell in vitro; or a cell in a multicellular organism; or in an extracellular fluid in a multicellular organism). Agents that can be tested for an effect on the level of an ROS in a cell include, but are not limited to, therapeutic agents; growth factors; neurotransmitters; anesthetics; hormones; metal ions; receptor agonists; receptor antagonists; and any other agent that can be administered to cells and/or multi-cellular organisms.

A subject compound can be administered to an individual via any number of modes and routes of administration. In some embodiments, a subject compound is administered systemically (e.g., via intravenous injection; via oral administration; etc.). In other embodiments, a subject compound is administered locally. A subject compound can be administered intravenously, intratumorally, peritumorally, orally, topically, subcutaneously, via intraocular injection, rectally, vaginally, or any other enteral or parenteral route of administration.

### Detection of an ROS in a Cell-Free Sample

The present disclosure provides a method of detecting an ROS (e.g., H₂O₂) in a cell-free sample in vitro. In some embodiments, a subject detection method involves contacting a subject compound with a cell-free sample in vitro; and detecting a signal generated by reaction of the compound with an ROS (e.g., H₂O₂) in the cell-free sample. In some embodiments, the cell-free sample is a biological sample.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Imaging Hydrogen Peroxide Production in Living Mice with a Chemoselective Bioluminescent Reporter

### Materials and Methods

### Synthesis of Reagents

Synthetic procedures for the synthesis of the compounds shown in Scheme 1 are described below.

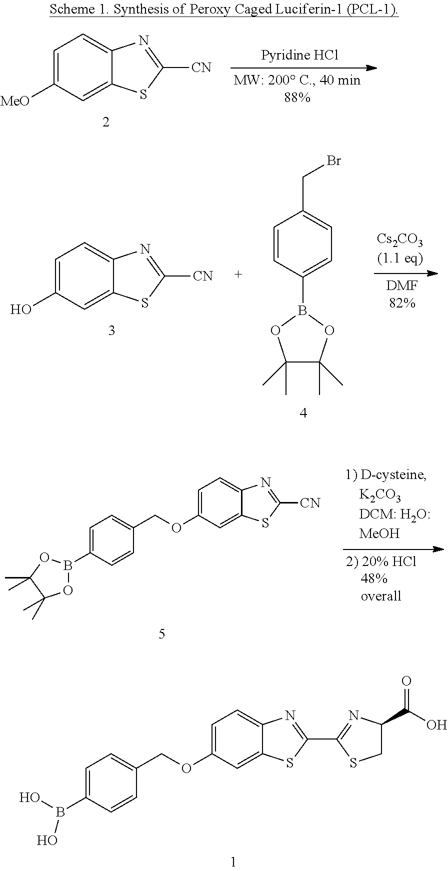

In Vitro Assays.

Millipore water was used to prepare all aqueous solutions. Measurements for in vitro tests were performed in 50 mM Tris buffer, pH 7.4, with 10 mM MgCl₂, 0.1 mM ZnCl₂, and 2 mM ATP at 37° C. Bioluminescent measurements for in vitro tests were recorded using a Molecular Devices SpectraMax M2 plate reader (Sunnyvale, Calif.). Samples for bioluminescent measurements were placed in white, opaque 96-well plates, which were purchased from Corning Inc. (Corning, N.Y.). ATP was purchased from MP Biomedicals (Solon, Ohio), and luciferase was purchased from Promega (Madison, Wis.).

Selectivity Tests.

Various reactive oxygen species (ROS, 100 μM) were administered to PCL-1 (5 μM) in the Tris buffer as follows. Hydrogen peroxide (H₂O₂), tert-butyl hydroperoxide (TBHP), and hypochlorite (OCl⁻) were delivered from 30%, 70%, and 6.15% aqueous solutions, respectively. Hydroxyl radical (.OH) and tert-butoxy radical (.OtBu) were generated by reaction of 1 mM Fe²⁺ with 100 μM H₂O₂ or TBHP, respectively. Nitric oxide (NO.) was delivered using PROLI NONOate. Superoxide (O₂⁻) was produced by xanthine oxidase (4.5×10⁻³ mg/100 μL) in the presence of hypoxanthine (2 mM) and catalase (0.4 mg/mL) or delivered from a 10 mM stock solution of potassium superoxide (KO₂) in dimethylsulfoxide (DMSO). Experiments with H₂O₂ and catalase were performed with 100 μM H₂O₂ and 0.4 mg/mL catalase. After each ROS was incubated with the caged luciferin for 5, 20, 40, or 60 minutes, 100 μL of the Tris buffer containing 100 μg/mL luciferase and 2 mM ATP was added to 100 μL of the PCL-1 solution.

Concentration Dependence.

To determine the dependence of PCL-1 signal on H₂O₂ concentrations, the probe was incubated in the Tris buffer (100 μL) with various concentrations of H₂O₂ for 60 minutes prior to the addition of Tris buffer (100 μL) containing 100 μg/mL luciferase and 2 mM ATP.

Ex Vivo Assays.

A Xenogen IVIS Spectrum instrument (Caliper Life Sciences, Hopkinton, Mass.) was used for bioluminescent imaging in all ex vivo experiments. LNCaP-luc cells (kindly provided by Chris Contag, Stanford University) were cultured in Dulbecco's Modified Eagle Medium (DMEM) containing 10% Fetal Bovine Serum (FBS). Cells were split twice per week. Prior to assaying, cells were passed and plated (4×10⁴ cells/well) in black 96-well plates with clear bottoms (Becton Dickinson and Company, Franklin Lakes, N.J.). Two days after being plated, once the cells were ca. 95% confluent, the medium was removed and PCL-1 (2.5% final DMSO concentration) and H₂O₂ (2.5-500 μM final concentrations) in DMEM (−FBS) were added. The plate was immediately imaged for 2 hours. For stimulation experiments, the cells (ca. 95 confluent) were incubated with paraquat (0 or 500 μM, 100 μL DMEM+10% FBS) for 24 hours prior to the addition of PCL-1 (50 μM) with or without catalase (1×10̂4 U/L).

In Vivo Experiments.

A Xenogen IVIS Spectrum instrument (Caliper Life Sciences, Hopkinton, Mass.) was used for bioluminescent imaging in all in vivo experiments. Phosphate Buffered Saline (PBS) was purchased from Thermo Fisher Scientific (Waltham, Mass.). Isoflurane was purchased from Phoenix Pharmaceuticals, Inc. (St. Joseph, Mo.), and sterile DMSO was purchased from Sigma-Aldrich (St. Louis, Mo.). Medical grade oxygen was purchased from Praxair (Danbury, Conn.).

Animals. FVB-luc (FVB-Tg(CAG-luc,-GFP)L2G85Chco/J) mice were obtained from UC Davis, and SHO mice were obtained from Charles River Labs. Mice were single or group-housed on a 12:12 light-dark cycle at 71° C. with free access to food and water. All studies were approved and performed according to the guidelines of the Animal Care and Use Committee of the University of California, Berkeley.

In Vivo Exogenous H₂O₂ Experiments.

Adult FVB-luc mice were anesthetized with isoflurane and injected (i.p.) with PCL-1 (0.5 μmol, 50 μL 1:1 DMSO:PBS), followed immediately by an i.p. injection of H₂O₂ (0.0375, 0.15, 0.6, and 2.4 μmol in 100 μL PBS). Control mice were injected with PCL-1 and 100 μL PBS.

In Vivo Antioxidant Experiments.

Adult FVB-luc mice were anesthetized with isoflurane and injected with N-acetylcysteine (0.2 mg, 100 μL PBS, pH 7-8), followed immediately by an i.p. injection of PCL-1 (5 μmol, 50 μL 1:1 DMSO:PBS). Control mice were injected with 100 μL PBS and PCL-1.

Mouse Tumor Model.

Tumor xenografts were created via i.p. injection of 3×10̂6 LNCaP-luc cells (100 μL, 1:1 PBS:Matrigel) in adult SHO mice. Tumor size was monitored by injecting 0.5-5 μmol D-lucifeirn (potassium salt, Gold Biotech., St. Louis, Mo.) in PBS. At least two weeks post-inoculation, tumors were injected with PCL-1, valeryl luciferin, testosterone propionate and N-acetylcysteine in various combinations to obtain the experimental data. On day one of the experiments, mice were injected with either PCL-1 (0.5 μmol) or valeryl luciferin (0.44 μmol) and imaged for 45 minutes using an IVIS spectrum. To determine the effect of testosterone on the tumors, the mice were injected with testosterone propionate (3 mg in 50 μL sesame oil) on day 2, followed 1.5 hours later by an injection of PCL-1 (0.5 μmol) or valeryl luciferin (0.44 μmol). Controls for the testosterone experiment were completed by injecting sesame oil (50 μL) on day 2, followed by the probe (0.5 μmol) or valeryl luciferin (0.44 μmol) 1.5 hours later. Additional experiments with N-acetylcysteine were completed by injecting the mice with testosterone propionate (3 mg in 50 μL sesame oil) on day 2, followed 1.5 hours later by injections of N-acetylcysteine (0.2 mg in 100 μL PBS) and PCL-1 (0.5 μmol). Day 2 injections were followed by 45 minutes of imaging.

Synthetic Materials and Methods.

Compounds 6 and 7 (Scheme 2) were synthesized according to literature procedures.⁷⁷ Chemicals were purchased from Sigma-Aldrich (St. Louis, Mo.), EMD Chemicals Inc. (Gibbstown, N.J.), Alfa Aesar (Ward Hill, Mass.), and Thermo Fisher Scientific (Waltham, Mass.) and were used as received. Column chromatography was performed using SiliaFlash P60 silica gel (40-63 microns) from Silicycle (Quebec, Canada). Analytical thin layer chromatography was performed using glass-backed SiO₂ TLC plates from Silicycle. High performance liquid chromatography was performed with a Varian DYNAMAX Microsorb C-18 preparative column (21.4×250 mm) with an attached DYNAMAX guard column on a Varian Pro Star system with a Varian UV-Vis detector, model 330. NMR spectra were obtained in deuterated solvents from Cambridge Isotope Laboratories (Cambridge, Mass.) on Bruker AV-300 or AV-400 spectrometers at the College of Chemistry NMR Facility at the University of California, Berkeley. All chemical shifts are reported in the standard δ notation of parts per million using the peaks of residual proton and carbon signals of the solvent as internal references. Low resolution Electrospray Ionization (ESI) mass spectral analyses were performed on an Agilent 6100 series single quad LC/MS system or an Agilent 7890A GC system with a 5975C inert MSD with a triple-axis detector. High-resolution fast atom bombardment (FAB) and ESI mass spectral analyses were performed by the College of Chemistry Mass Spectrometry Facility at the University of California, Berkeley.

### 2-Cyano-6-hydroxybenzothiazole (3)

Compound 1 was synthesized using a method modified from the literature.⁶⁴ Pyridine hydrochloride (1.0 g, 8.65 mmol) and 2-cyano-6-methoxybenzothiazole, 2, (0.5 g, 2.63 mmol) were added to a 5 mL microwave flask with a stirbar. Nitrogen gas was added to the reaction vessel immediately before it was shut. The flask was heated to 200° C. using a power level of 150 W for 40 minutes in a Biotage microwave synthesizer. The reaction was stirred at 600 rpm. The reaction was cooled and neutralized with sodium bicarbonate. During neutralization, the crude product precipitated from the solution as a yellow solid. The precipitate was filtered, and the filtrate was washed three times with ethyl acetate. Combination of the crude product from the ethyl acetate washes and the yellow precipitate and purification on a silica column (70:30 hexanes:ethyl acetate, dry loaded) yielded 408.7 mg (88%) of the pure product. ¹H NMR (300 MHz, CD₃OD): δ 7.13 (1H, dd, J=9 Hz), 7.36 (1H, d, J=2.1 Hz), 7.95 (1H, d, J=9 Hz). LRESI-MS: calculated for [C₈H₄N₂OS] 176.0. found 176.1.

### 6-(4-(4,4,5,5-Tetramethyl-1,3,2-dioxaborolan-2-yl)benzyloxy)benzo[d]thiazole-2-carbonitrile (5)

Compounds 3 (300 mg, 1.7 mmol) and 4 (505.7 mg, 1.7 mmol) were dissolved in 30 mL dry DMF prior to the addition of cesium chloride (610.25 mg, 1.87 mmol). The mixture was stirred at 60° C. for 45-50 min before it was allowed to cool to room temperature. 100 mL ethyl acetate was added to the reaction, and the organic phase was washed three times with deionized water. The aqueous layers were combined and washed three times with ethyl acetate. All of the organic layers were combined, washed twice with brine, dried over sodium sulfate, and concentrated. The crude material was purified on a silica column (90:10 hexanes:ethyl acetate, dry loaded) to give 547.4 mg (82%) of the pure product. ¹H NMR (400 MHz, CDCl₃): δ 1.36 (1H, s), 5.21 (2H, s), 7.32 (1H, d, J=8.8 Hz), 7.40 (1H, s), 7.45 (2H, d, J=7.6 Hz), 7.86 (2H, d, J=7.2 Hz), 8.09 (1H, d, J=9.2). LRESI-MS: calculated for [C₂₁H₂₂BN₂O₃S]⁺393.1. found 393.1.

### (S)-2-(6-(4-Boronobenzyloxy)benzo[d]thiazol-2-yl)-4,5-dihydrothiazole-4-carboxylic acid (1)

D-cysteine hydrochloride (58.7 mg, 0.334 mmol) and potassium carbonate (50.36 mg, 0.364 mmol) were dissolved in 2 mL N₂-sparged DI H₂O under an N₂ atmosphere. 5 mL N₂-sparged dichloromethane was added to the flask, followed by 5 (100 mg, 0.304 mmol). After 5 dissolved, the flask was charged with 7.6 mL N₂-sparged methanol. The flask was monitored to ensure that no precipitate formed while the methanol was added. The reaction was stirred vigorously for ten minutes before 3 mL N₂-sparged DI H₂O were added to the flask. The DCM and MeOH were removed from the flask under low pressure prior to the addition of 25 mL of a N₂-sparged 20% aqueous HCl solution. A yellow precipitate formed immediately upon addition of the HCl solution. The mixture was stirred for 15-20 minutes and the precipitate was filtered and washed with DI H₂O until the pH became neutral. The crude material was purified using HPLC (H₂O:MeOH, 40-100% MeOH over 45 minutes) to give 58.9 mg (47%) pure product. ¹H NMR (400 MHz, CD₃OD): δ 3.78 (2H, dd, J=9.2, 2.8 Hz), 5.22 (2H, s), 5.40 (1H, t, J=9.2), 7.26 (1H, dd, J=9.2, 2.6 Hz), 7.48 (2H, d, J=6.4 Hz), 7.63 (1H, d, J=2.4 Hz), 7.67 (1H, br), 7.78 (1H, br), 7.98 (1H, d, J=9.2). ¹³C NMR (100 MHz, CD₃OD): δ 34.51, 70.11, 78.22, 104.87, 117.35, 124.43, 126.35, 133.47, 137.61, 147.56, 158.22, 158.55, 166.10, 172.04. HRESI-MS: calculated for [C₁₈H₁₄BN₂O₅S₂]⁻ 413.0437. found 413.0445.

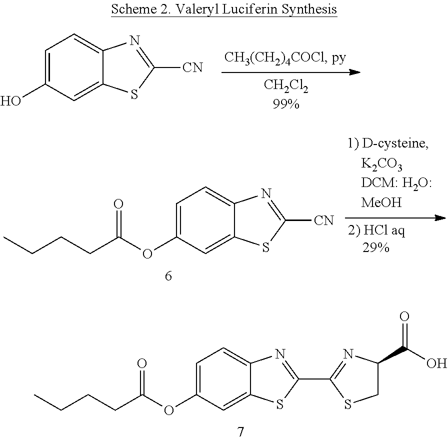 [SMILES: N#Cc1nc2ccc(O)cc2s1 ; CCCCC(=O)Oc1ccc2nc(C#N)sc2c1 ; CCCCC(=O)Oc1ccc2nc(C3=N[C@@H](C(=O)O)CS3)sc2c1]

### Results

### Design and Synthesis of Peroxy Caged Luciferin (PCL-1)

Desirable properties for an effective H₂O₂ reporter in living animals include selectivity for H₂O₂ over biologically relevant ROS, a good signal-to-noise contrast ratio, high efficiency signal production, and deep tissue penetration. In addition, practical molecular imaging probes for use in whole organisms should be readily transported in vivo, minimally invasive, and non-toxic. The firefly luciferin/luciferase bioluminescent reporter system was chosen as a platform for creating new in vivo H₂O₂ imaging agents as it meets all of the aforementioned chemical and biological criteria. In particular, the firefly luciferin substrate is a non-toxic small molecule that easily enters the blood stream, produces deep-tissue penetrable signal in all organs of mice, and is metabolized within hours.

Efforts were made to develop a bioluminescent H₂O₂ reporter in which an appropriately caged luciferin that is unreactive toward the luciferase enzyme could be unmasked by a selective H₂O₂-mediated cleavage process to generate luciferin and subsequently trigger the catalytic bioluminescent luciferin/luciferase reaction. Related strategies have been employed for assaying enzyme activity⁵⁷⁻⁶⁶ and cell membrane transporter efficiency.⁶⁷⁻⁶⁹ In particular, alkylation of firefly luciferin at the phenolic position prevents signal production from firefly luciferin even when luciferin's reactive carboxylic acid moiety remains unaltered,⁷⁰ and previous work from our laboratory has shown that the chemoselective deprotection of boronate esters to phenols offers a useful reaction-based method for detecting H₂O₂ over other ROS.^{38, 39, 41-47} Based on these considerations, the peroxide-sensitive probe Peroxy Caged Luciferin-1 (PCL-1, FIG. 1) was designed by attaching an aryl boronate to firefly luciferin through a self-immolative benzylic linker. In the absence of H₂O₂, PCL-1 is not an active substrate for the luciferin enzyme and hence does not generate light output. Addition of H₂O₂ triggers cleavage of the boronate benzyl ether to release free luciferin, which can then react with luciferase and produce a bioluminescent readout. The synthesis of PCL-1 is depicted in Scheme 1. Deprotection of 2-cyano-6-methoxybenzothiazole 2 using pyridinium chloride furnishes phenol 3. Alkylation of 3 with benzyl bromide 4 places the linker and boronate on the benzothiazole ring to give 5. Condensation of benzothiazole 5 with D-cysteine and subsequent acid workup affords the final PCL-1 probe 1. The overall yield for this reaction sequence is 35%.

### Chemoselective and Concentration-Dependent Bioluminescence Detection of H₂O₂

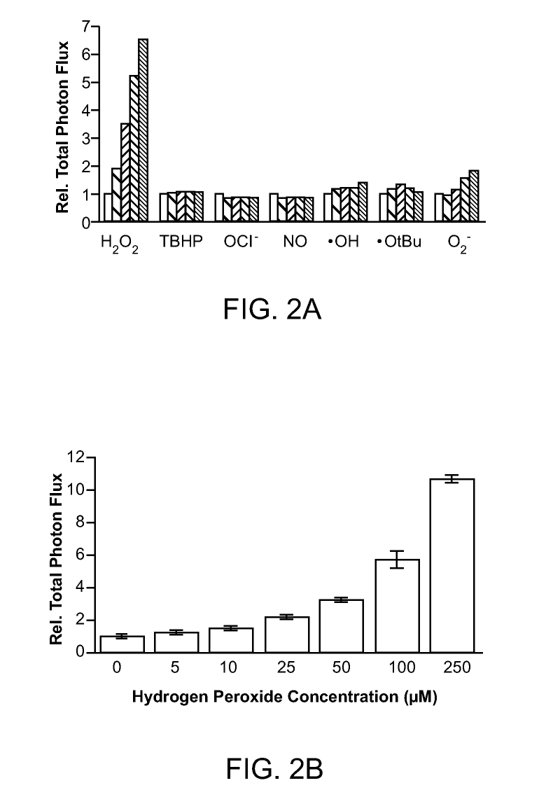

Initial experiments tested the ability of PCL-1 to detect H₂O₂ in a selective and concentration-dependent manner. First, an in vitro bioluminescent assay was performed, using purified firefly luciferase enzyme to evaluate the ROS selectivity of PCL-1. PCL-1 was incubated with various ROS for 5-60 minutes followed by addition of firefly luciferase, and then light production was measured over 45 minutes to determine whether luciferin was produced during the ROS incubation period. The total normalized photon flux was calculated by dividing each total photon flux by the zero timepoint total photon flux for each run to allow a direct comparison between various ROS. Whereas addition of H₂O₂ showed an approximately seven-fold increase in bioluminescent signal over an hour, there was little to no increase in signal when the boronate probe was reacted with the other ROS (FIG. 2).

After confirming the selectivity of PCL-1 for H₂O₂ over other biologically relevant ROS, the responsiveness of PCL-1 to alterations in peroxide levels was examined. PCL-1 was incubated with various concentrations of H₂O₂ for 60 minutes followed by addition of firefly luciferase, and then light production was measured. This assay establishes that the bioluminescent reaction readout is linearly dependent on the concentration of H₂O₂ over a two order-of-magnitude range from 5 to 250 μM (FIG. 2). Taken together, the high specificity of the PCL-1 reporter system for H₂O₂ over other biological ROS, as well as its sensitivity to a physiologically relevant low micromolar detection limit and dose-dependent response for this reactive oxygen metabolite, are necessary features of a practical probe that can detect changes in H₂O₂ levels in living cells and in living animals in the presence of oxidative or reductive stimuli.

### FIGS. 2A and 2B. Selective and Concentration Dependent Bioluminescent Detection of H₂O₂ by PCL-1.

(a) Total bioluminescent signal, integrated over 45 minutes, from PCL-1 (5 μM; open bars) incubated with various ROS (100 μM) for 0, 5, 20, 40, and 60 minutes (hatched bars). (b) Total bioluminescent signal, integrated over 45 minutes, from 5 μM PCL-1 incubated for 1 h with increasing concentrations of H₂O₂ (0-250 μM). In order to quantify free luciferin formation in a and b, 100 ug/mL luciferase in 50 mM Tris buffer with 10 mM MgCl₂, 0.1 mM ZnCl₂, and 2 mM ATP (pH 7.4) was added to the PCL-1 plus ROS solutions.

### PCL-1 can Visualize Changes in H₂O₂ Levels in Living Cells by Bioluminescence Imaging

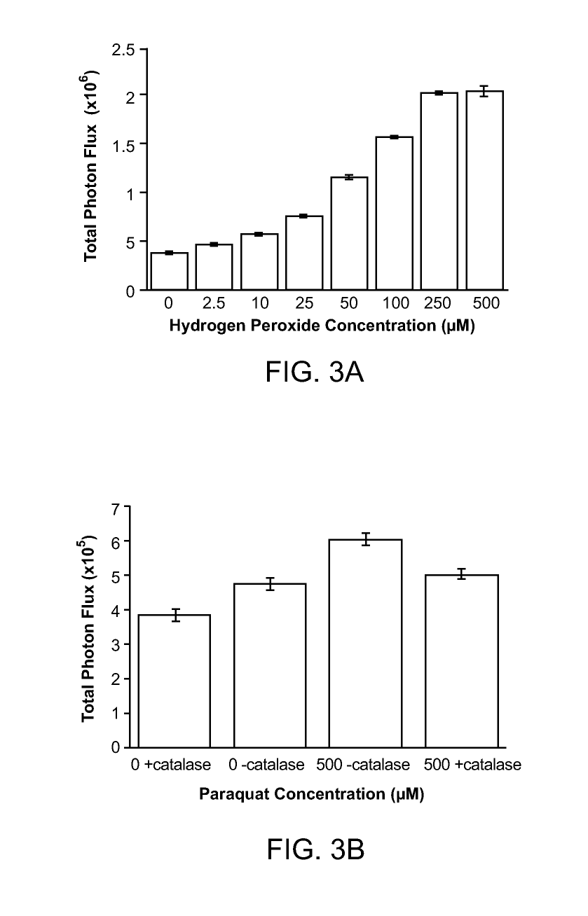

With data showing that PCL-1 is a selective and sensitive bioluminescent reporter for H₂O₂ in aqueous solution, this system was applied to imaging changes in H₂O₂ levels in cell culture. Initial experiments focused on live-cell assays with exogenous H₂O₂ addition to ensure that the presence of cells did not interfere with production of the H₂O₂-dependent bioluminescent signal. To achieve this goal, PCL-1 and H₂O₂ were added to LNCaP-luc cells stably transfected with the firefly luciferase gene. In the absence of H₂O₂, the bioluminescent signal from the cells was negligible. In contrast, addition of 2.5 to 500 μM H₂O₂ triggers a linear increase, up to 250 μM, in the total observed photon flux (FIG. 3). The 2.5 μM detection limit for H₂O₂ is well within estimated concentration ranges of endogenously produced H₂O₂ fluxes.

The foregoing cell experiments demonstrate that the presence of cells does not interfere with H₂O₂-triggered bioluminescent signal production from PCL-1 with exogenous H₂O₂ addition. To determine whether PCL-1 could detect endogenously produced H₂O₂ in living cells, LNCaP-luc cells were incubated with 500 μM paraquat for 24 hours, as previous work establishes that paraquat triggers elevations in intracellular H₂O₂ through disruption of the mitochondrial electron transport chain. Following paraquat stimulation, the LNCaP-luc cells were loaded with PCL-1 and the subsequent bioluminescent signal was detected. Paraquat-treated cells show patently higher levels of PCL-1 derived luminescence compared to unstimulated control cells (FIG. 3).

Because luciferin is a cell-permeable small molecule, two possible modes of action for PCL-1 are (i) reaction of this probe with H₂O₂ in the extracellular medium followed by cellular uptake of the free luciferin product, or (ii) reaction of PCL-1 with intracellular H₂O₂ to generate luciferin within cells. To probe whether PCL-1 was reacting with H₂O₂ in the intra- or extracellular space, catalase was added as a cell-impermeable scavenger for extracellular H₂O₂ with paraquat stimulation. Interestingly, it was observed that the addition of catalase with paraquat causes a decrease in bioluminescence signal detected from the cells compared to addition of paraquat alone; however, the signal did not decrease to the level of background signal of cells treated only with catalase in the absence of paraquat (FIG. 3). These data indicate that PCL-1 is capable of interacting with both extracellular and intracellular H₂O₂ pools. Moreover, because treatment with the antioxidant catalase reduces the level of PCL-1 bioluminescence of cells compared to untreated specimens, the data also suggest that PCL-1 is sensitive enough to detect basal levels of H₂O₂ that are endogenously produced without addition of external H₂O₂, highlighting the potential utility of PCL-1 for in vivo studies.

FIGS. 3A and 3B. Bioluminescent Signal from PCL-1 Added to LNCaP-Luc Cells. (a)

Total photon flux, integrated over 2 h, from LNCaP-luc cells with PCL-1 (50 μM) and H₂O₂ (2.5-500 μM) in DMEM. All values show statistically significant differences (p<0.001) except between 250 and 500 μM H₂O₂. (b) Total photon flux, integrated over 2 h, from LNCaP-luc cells incubated with paraquat (0 or 500 μM) for 24 h, followed by addition of PCL-1 (50 μM)±catalase (1×10̂4 U/L). All values show statistically significant differences (p<0.01), except between 0 μM paraquat without catalase and 500 μM paraquat with catalase.

Molecular Imaging of H₂O₂ Fluxes in Living Animals with FVB-Luc Mice

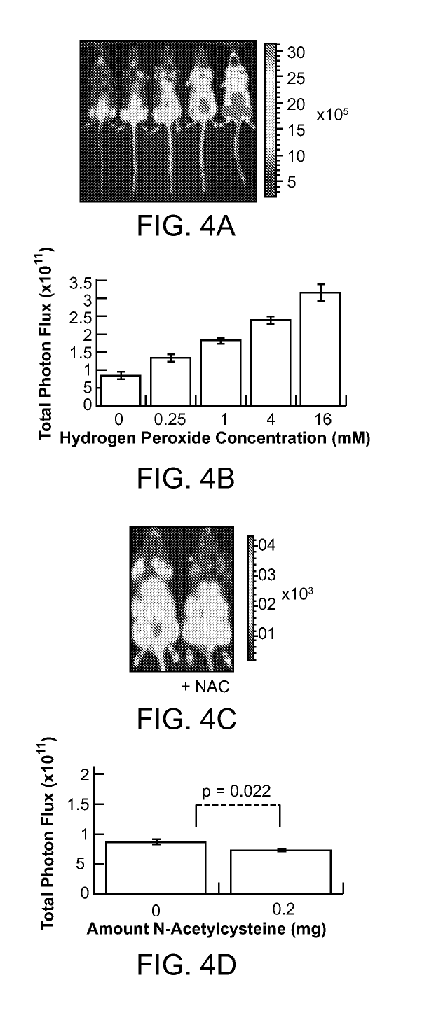

PCL-1 was applied to molecular imaging of H₂O₂ in living animals. Initial studies utilized FVB-luc mice that ubiquitously express firefly luciferase along with exogenous peroxide addition. Several concentrations of H₂O₂ were injected into the intraperitoneal cavity of the mice with subsequent i.p. injection of PCL-1, and the bioluminescence from these living animals was imaged in real-time using a CCD camera. Monitoring the integrated total photon flux for each mouse reveals a dose-dependent increase in signal as a function of the H₂O₂ concentration (FIG. 4). Interestingly, mice that were treated only with PCL-1 with no added peroxide also show modest but measurable bioluminescence, suggesting that PCL-1 may be detecting basal levels of H₂O₂ produced in these living animals. To determine whether this emission signal was due in part to the detection of endogenous H₂O₂, PCL-1 was injected into FVB-luc mice in the presence and absence of the antioxidant N-acetylcysteine (NAC). The NAC-treated animals exhibited a significantly reduced bioluminescent signal compared to vehicle control animals, establishing that PCL-1 is sensitive enough to visualize basal levels of H₂O₂ in living animals without external stimulation of peroxide production.

FIGS. 4A-D. Bioluminescent Signal from PCL-1 in FVB-Luc Mice. (a)

Representative image (30 min post-injection) for mice injected with PCL-1 (i.p., 0.5 μmol, 50 μL) immediately prior to injection of H₂O₂ (i.p., 0-16 mM, 100 μL). All values show statistically significant differences (p<0.01). (b) Total photon flux, integrated over one hour, for mice injected with PCL-1±H₂O₂. Each bar represents the average signal from five mice. (c) Representative image (12 min post-injection) for mice injected with PCL-1 (i.p., 0.5 μmol, 50 μL) immediately following N-acetylcysteine (NAC) (i.p., 0-0.2 mg, 100 μL). (d) Total photon flux, integrated over one hour, for mice injected with PCL-1±NAC. Each bar represents the average signal from three mice.

### Detection of Endogenous H₂O₂ in a Prostate Tumor Model

After establishing that the H₂O₂-mediated boronate deprotection of PCL-1 provides a selective and sensitive platform for real-time imaging of H₂O₂ in water, in living cells, and in living mice, this bioluminescent reporter was applied to studies of H₂O₂ physiology at an in vivo level. H₂O₂ plays a role in the development and progression of cancer; and recent reports suggest that tumor cells produce an elevated level of H₂O₂ compared to noncancerous cells, and that this ROS increase is correlated with cancer cell growth and malignancy. Initial experiments focused on prostate cancer using the androgen-sensitive prostate cancer cell model (LNCaP). In dissociated cell culture, LNCaPs respond to testosterone by increasing their proliferation rate.⁷¹⁻⁷³ and elevating their ROS production,⁷⁴ suggesting a link between oxidative stress and development of this disease. However, given that the probes used to detect oxidant production were not specific to any particular ROS and that the environment that tumor cells experience in vivo is greatly different from dissociated cell culture conditions with variances in oxygen level, nutrient supply, and acidity,⁷⁵ the application of tools to probe specific ROS molecules like H₂O₂ in the context of a living animal is critical to elucidating the relationships between redox biology and cancer.

To study the stimulatory effects of testosterone and H₂O₂ production in prostate cancer in living animals, an intraperitoneal (i.p.) LNCaP-luc tumor xenograft model was developed in immunodeficient severe combined immunodeficiency hairless outbred (SHO) mice. To determine the baseline levels of H₂O₂ generation in the tumors as well as alterations in H₂O₂ fluxes over 24 hours, mice were injected with PCL-1 on Day 1 and with sesame oil, a vehicle used for all experiments, and PCL-1 after 24 hours on Day 2. These experiments clearly showed that there is no change in basal bioluminescent signal from the mice over the time course of these experiments. The effects of testosterone (in the form of testosterone propionate) on H₂O₂ production were tested within the prostate cancer tumors. For these experiments, mice were injected with PCL-1 on Day 1 and the baseline signal was measured. On Day 2, the mice were injected either with testosterone propionate and then PCL-1, or empty vehicle and PCL-1. Mice treated with testosterone propionate show an approximately 41% increase in total photon flux compared to vehicle control mice. These data suggest that LNCaP-luc tumors produce elevated levels of H₂O₂ in vivo upon testosterone stimulation.

To ensure that the observed signal enhancement from testosterone stimulation of the LNCaP tumors was due to an increase in H₂O₂ production and not a result of non-specific cellular and metabolic changes, a non-ROS responsive control compound, valeryl luciferin (7, Scheme 2), was utilized in identical experiments outlined above for PCL-1. This esterase-cleavable luciferin was chosen as the control compound instead of firefly luciferin because the peak for signal produced by luciferin in LNCaP-luc cells is reached prior to the first imaging timepoint (<1 min after injection). In contrast, as valeryl luciferin requires removal of the valeryl ester prior to light production, the signal peak is shifted to later timepoints and can be detected within the timeframe of the imaging experiments. No change was observed in bioluminescent signal from valeryl luciferin from Day 1 to Day 2 when mice were injected with vehicle alone or vehicle plus testosterone on the second day. These results indicate that testosterone does not alter the expression of firefly luciferase in the LNCaP-luc cells nor change the interactions between the luciferin derivatives and these tumor cells, which further validates that PCL-1 is imaging changes in tumor production of H₂O₂ upon testosterone stimulation.

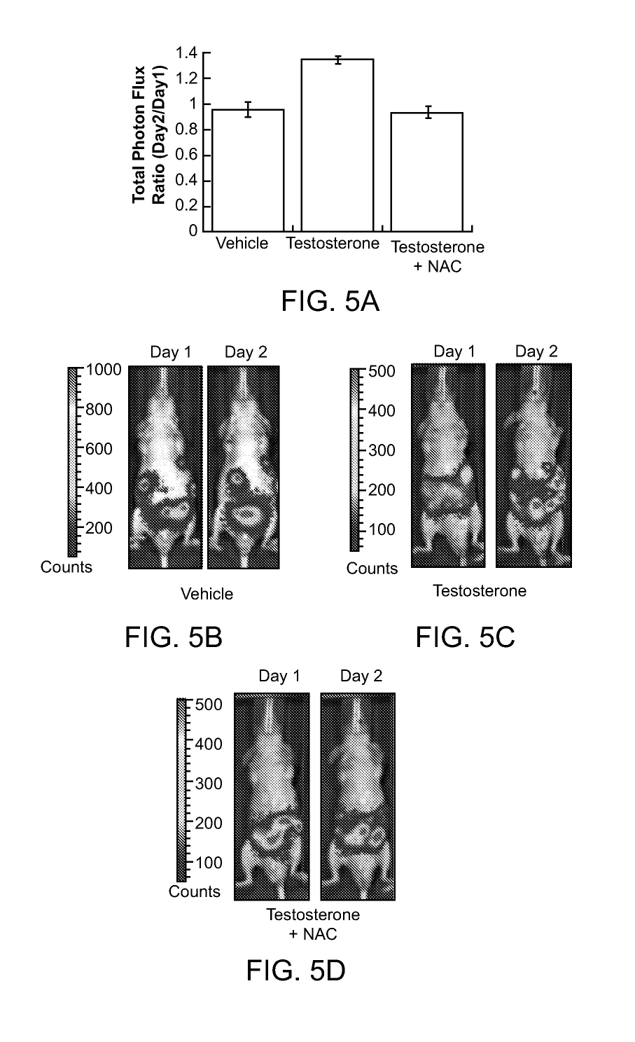

In a final set of control experiments to confirm that PCL-1 is detecting a testosterone-triggered increase in tumor H₂O₂ production, NAC was used as a general chemical scavenger for H₂O₂. These experiments were performed by injecting mice on Day 2 with testosterone propionate, followed by serial application of NAC and PCL-1. As shown in FIG. 5, NAC treatment causes a reduction in bioluminescent signal in testosterone-stimulated animals back to baseline levels, with light production comparable to vehicle control PCL-1 tumor xenografts. Taken together, the collective data establishes that androgen-sensitive prostate tumors respond to the proliferation signal of testosterone in vivo by elevating their production of H₂O₂.

FIGS. 5A-D. Bioluminescent Signal from SHO Mice with LNCaP-Luc Tumors. (a)

Ratios of total photon fluxes for mice injected with PCL-1 (i.p., 0.5 μmol) on day 1 and PCL-1 (i.p., 0.5 μmol) plus (b) the vehicle (sesame oil, 50 μL), (c) testosterone (3 mg, 50 μL sesame oil) or (d) testosterone (3 mg, 50 μL sesame oil) and N-acetylcysteine NAC (0.2 mg, 100 μL PBS) on day 2. Sesame oil and testosterone were injected 1.5 h prior to PCL-1 and NAC was injected immediately prior to PCL-1 on day 2. All bars in a represent data from 5 mice. Representative images from one mouse in each experiment are shown (b-d). The differences in bioluminescence observed from mice treated with testosterone and either vehicle only or testosterone plus NAC are statistically significant (p<0.001).

### REFERENCES

1. Sundaresan, M., Yu, Z. X., Ferrans, V. J., Irani, K., Finkel, T. Requirement for generation of H₂O₂ for platelet-derived growth factor signal transduction. Science 270, 296-299 (1995).
2. Bae, Y. S., Kang, S. W., Seo, M. S., Baines, I. C., Tekle, E., Chock, P. B., Rhee, S. G. Epidermal growth factor (EGF)-induced generation of hydrogen peroxide: role in EGF receptor-mediated tyrosine phosphorylation. J. Biol. Chem. 272, 217-221 (1997).
3. Wood, Z. A., Poole, L. B., Karplus, P. A. Peroxiredoxin evolution and the regulation of hydrogen peroxide signaling. Science 300, 650-653 (2003).
4. Woo, H. A., Chae, H. Z., Hwang, S. C., Yang, K. S., Kang, S. W., Kim, K., Rhee, S. G. Reversing the inactivation of peroxiredoxins caused by cysteine sulfinic acid formation. Science 300, 653-656 (2003).
5. Avshalumov, M. V., Rice, M. E. Activation of ATP-sensitive K⁺ (K_{ATP}) channels by H₂O₂ underlies glutamate-dependent inhibition of striatal dopamine release. Proc. Natl. Acad. Sci. USA 100, 11729-11734 (2003).
6. Lambeth, J. D. Nox enzymes and the biology of reactive oxygen. Nat. Rev. Immunol. 4, 181-189 (2004).
7. Rhee, S. G. H₂O₂, a necessary evil for cell signaling. Science 312, 1882-1883 (2006).
8. Stone, J. R., Yang, S. Hydrogen peroxide: a signaling messenger. Antiox. Redox Signal. 8, 243-270 (2006).
9. Leto, T. L., Geiszt, M. Role of Nox family NADPH oxidases in host defense. Antioxid. Redox Signaling 8, 1549-1561 (2006).
10. Bedard, K., Krause, K.-H. The Nox family of ROS-generating NADPH oxidases: physiology and pathophysiology. Physiol. Rev. 87, 245-313 (2007).
11. D'AutrEaux, B., Toledano, M. B., ROS as signaling molecules: mechanisms that generate specificity in ROS homeostasis. Nat. Rev. Mol. Cell. Biol. 8, 813-824 (2007).
12. Giorgio, M., Trinei, M., Migliaccio, E., Pelicci, P. G. Hydrogen peroxide: a metabolic by-product or a common mediator of ageing signals? Nat. Rev. Mol. Cell. Biol. 8, 722-728 (2007).
13. Miller, E. W., Chang, C. J. Fluorescent probes for nitric oxide and hydrogen peroxide in cell signaling. Curr. Opin. Chem. Biol. 11, 620-625 (2007).
14. Veal, E. A., Day, A. M., Morgan, B. A. Hydrogen peroxide sensing and signaling. Mol. Cell. 26, 1-14 (2007).
15. Poole, L. B., Nelson, K. J. Discovering mechanisms of signaling-mediated cysteine oxidation. Curr. Opin. Chem. Biol. 12, 18-24 (2008).
16. Janssen-Heininger, Y. M. W., Mossman, B. T., Heintz, N. H., Forman, H. J., Kalyanaraman, B., Finkel, T., Stamler, J. S., Rhee, S. G., van der Vliet, A. Redox-based regulation of signal transduction: principles, pitfalls, and promises. Free Radical Biol. Med. 45, 1-17 (2008).
17. Winterbourn, C. C. Reconciling the chemistry and biology of reactive oxygen species. Nat. Chem. Biol. 4, 278-286 (2008).
18. Bao, L., Avshalumov, M. V., Patel, J. C., Lee, C. R., Miller, E. W., Chang, C. J., Rice, M. E. Mitochondria are the source of hydrogen peroxide for dynamic brain-cell signaling. J. Neurosci. 29, 9002-9010 (2009).
19. Kim, J. S., Huang, T. Y., Bokoch, G. M. Reactive oxygen species regulate a slingshot-cofilin activation pathway. Mol. Biol. Cell 20, 2650-2660 (2009).
20. Niethammer, P., Grabher, C., Look, A. T., Mitchison, T. J. A tissue-scale gradient of hydrogen peroxide mediates rapid wound detection in zebrafish. Nature 459, 996-999 (2009).
21. Paulsen, C. E., Carroll, K. S. Orchestrating redox signaling networks through regulatory cysteine switches. ACS Chem. Biol. 5, 47-62 (2010).
22. Woo, H. A., Yim, S. H., Shin, D. H., Kang, D., Yu, D. Y., Rhee, S. G. Inactivation of peroxiredoxin I by phosphorylation allows localized H₂O₂ accumulation for cell signaling. Cell 140, 517-528 (2010).
23. Finkel, T., Serrano, M., Blasco, M. A. The common biology of cancer and ageing. Nature 448, 767-774 (2007).
24. Ishikawa, K., Takenaga, K., Akimoto, M., Koshikawa, N., Yamaguchi, A., Imanishi, H., Nakada, K., Honma, Y., Hayashi, J.-I. ROS-generating mitochondrial DNA mutations can regulate tumor cell metastasis. Science 320, 661-664 (2008).
25. Goetz, M. E., Luch, A. Reactive species: a cell damaging rout assisting to chemical carcinogens. Cancer Lett. 266, 73-83 (2008).
26. Rossi, D. J., Jamieson, C. H. M., Weissman, I. L. Stem cells and the pathways to aging and cancer. Cell 132, 681-696 (2008).
27. Houstis, N., Rosen, E. D., Lander, E. S. Reactive oxygen species have a causal role in multiple forms of insulin resistance. Nature 440, 944-948 (2006).
28. Jay, D., Hitomi, H., Griendling, K. K. Oxidative stress and diabetic cardiovascular complications. Free Radical Biol. Med. 40, 183-192 (2006).
29. Looi, Y. H., Grieve, D. J., Siva, A., Walker, S. J., Anilkumar, N., Cave, A. C., Marber, M., Monaghan, M. J., Shah, A. M. Involvement of Nox2 NADPH oxidase in adverse cardiac remodeling after myocardial infarction. Hypertension 51, 319-325 (2008).
30. Nistico, R., Piccirilli, S., Cucchiaroni, M. L., Armogida, M., Guatteo, E., Giampa, C., Fusco, F. R., Bernardi, G., Nistico, G., Mercuri, N. B. Neuroprotective effect of hydrogen peroxide on an in vitro model of brain ischaemia. Br. J. Pharmacol. 153, 1022-1029 (2008).
31. Korge, P., Ping P., Weiss, J. N. Reactive oxygen species production in energized cardiac mitochondria during hypoxia/reoxygenation. Circ. Res. 103, 873-880 (2008).
32. Ushio-Fukai, M., Urao, N. Novel role of NADPH oxidase in angiogenesis and stem/progenitor cell function. Antioxid. Redox Signaling 11, 2517-2533 (2009).
33. Ardanaz, N., Yang, X.-P., Cifuentes, M. E., Haurani, M. J., Jackson, K. W., Liao, T.-D., Carretero, O. A., Pagano, P. J. Lack of glutathione peroxidase 1 accelerates cardiac-specific hypertrophy and dysfunction in angiotensin II hypertension. Hypertension 55, 116-123 (2010).
34. Barnham, K. J., Masters, C. L., Bush, A. I. Neurodegenerative diseases and oxidative stress. Nat. Rev. Drug Discov. 3, 205-214 (2004).
35. Lin, M. T., Beal, M. F. Mitochondrial dysfunction and oxidative stress in neurodegenerative diseases. Nature 443, 787-795 (2006).
36. DiMauro, S., Schon, E. A. Mitochondrial disorders in the nervous system. Annu. Rev. Neurosci. 31, 91-123 (2008).
37. Maeda, H., Fukuyasu, Y., Yoshida, S., Fukuda, M., Saeki, K., Matsuno, H., Yamauchi, Y., Yoshida, K., Hirata, K., Miyamoto, K. Fluorescent probes for hydrogen peroxide based on a non-oxidative mechanism. Angew. Chem. Int. Ed. 43, 2389-2391 (2004).
38. Chang, M. C. Y., Pralle, A., Isacoff, E. Y., Chang, C. J. A selective, cell-permeable optical probe for hydrogen peroxide in living cells. J. Am. Chem. Soc. 126, 15392-15393 (2004).
39. Miller, E. W., Albers, A. E., Pralle, A., Isacoff, E. Y., Chang, C. J. Boronate-based fluorescent probes for imaging cellular hydrogen peroxide. J. Am. Chem. Soc. 127, 16652-16659 (2005).
40. Soh, N. Recent advances in fluorescent probes for the detection of reactive oxygen species. Anal. Bioanal. Chem. 386, 532-543 (2006).
41. Albers, A. E., Okreglak, V. S., Chang, C. J. A FRET-based approach to ratiometric fluorescence detection of hydrogen peroxide. J. Am. Chem. Soc. 128, 9640-9641 (2006).
42. Miller, E. W., Tulyathan, O., Isacoff, E. Y., Chang, C. J. Molecular imaging of hydrogen peroxide produced for cell signaling. Nat. Chem. Biol. 3, 263-267 (2007).
43. Srikun, D., Miller, E. W., Domaille, D. W., Chang, C. J. An ICT-based approach to ratiometric fluorescence imaging of hydrogen peroxide produced in living cells. J. Am. Chem. Soc. 130, 4596-4597 (2008).
44. Dickinson, B. C., Chang, C. J. A targetable fluorescent probe for imaging hydrogen peroxide in the mitochondria of living cells. J. Am. Chem. Soc. 130, 9638-9639 (2008).
45. Albers, A. E., Dickinson, B. C., Miller, E. W., Chang, C. J. A red-emitting naphthofluorescein-based fluorescent probe for selective detection of hydrogen peroxide in living cells. Bioorg. Med. Chem. Lett. 18, 5948-5950 (2008).
46. Srikun, D., Albers, A. E., Nam, C. I., Iavarone, A. T., Chang, C. J. Organelle-targetable fluorescent probes for imaging hydrogen peroxide in living cells vie SNAP-tag protein labeling. J. Am. Chem. Soc. 132, 4455-4465 (2010).
47. Dickinson, B. C., Huynh, C., Chang, C. J. A palette of fluorescent probes with varying emission colors for imaging hydrogen peroxide signaling in living cells. J. Am. Chem. Soc. 132, 5906 (2010).
48. Oushiki, D., Kojima, H., Terai, T., Arita, M., Hanaoka, K., Urano, Y., Nagano, T. Development d application of a near-infrared fluorescence probe for oxidative stress based on differential reactivity of linked cyanine dyes. J. Am. Chem. Soc. 132, 2795-2801 (2010).
49. Belousov, V. V., Fradkov, A. F., Lukyanov, K. A., Staroverov, D. B., Shakhbazov, K. S., Terskikh, A. V., Lukyanov, S. Genetically encoded fluorescent indicator for intracellular hydrogen peroxide. Nature Methods 3, 281-286 (2006).
50. Gross, S., Gammon, S. T., Moss, B. L., Rauch, D., Harding, J., Heinecke, J. W., Ratner, L., Piwnica-Worms, D. Bioluminescence imaging of myeloperoxidase activity in vivo. Nat. Medicine 15, 455-461 (2009).
51. Kielland, A., Blom, T., Nandakumar, K. S., Holmdahl, R., Blomhoff, R., Carlsen, H. In vivo imaging of reactive oxygen and nitrogen species in inflammation using the luminescent probe L-012. Free Radical Biol. Med. 47, 760-766 (2009).

52. Lee, D., Khaja, S., Velasquez-Castano, J. C., Dasari, M., Sun, C., Petros, J., Taylor, W. R., Murthy, N. In vivo imaging of hydrogen peroxide with chemiluminescent nanoparticles. Nat. Mater. 6, 765-769 (2007).
53. Seliger, H. H., McElroy, W. D. Spectral emission and quantum yield of firefly bioluminescence. Arch. Biochem. Biophys. 88, 136-141 (1960).
54. Ando, Y., Niwa, K., Yamada, N., Enomoto, T., Irie, T., Kubota, H., Ohmiya, Y., Akiyama, H. Firefly bioluminescence quantum yield and color change by pH-sensitive green emission. Nat. Photonics 2, 44-47 (2008).
55. Zhao, H., Doyle, T. C., Coquoz, 0., Kalish, F., Rice, B. W., Contag, C. H. Emission spectra of bioluminescent reporters and interaction with mammalian tissue determine the sensitivity of detection in vivo. J. Biomed. Opt. 10, 041210 (2005).
56. Prescher, J. A., Contag, C. H. Guided by the light: visualizing biomolecular processes in living animals with bioluminescence. Curr. Opin. Chem. Biol. 14, 80-89 (2010).
57. Miska, W., Geiger, R. Synthesis and characterization of luciferin derivatives for use in bioluminescence enhanced enzyme immunoassays. J. Clin. Chem. Clin. Biochem. 25, 24-30 (1987).
58. Geiger, R., Schneider, E., Wallenfels, K., Miska, W. A new ultrasensitive bioluminogenic enzyme substrate for β-galactosidase. Biol. Chem. Hoppe-Seyler 373, 1187-1191 (1992).
59. Monsees, T., Miska, W., Geiger, R. Synthesis and characterization of a bioluminogenic substrate for α-chymotrypsin. Anal. Biochem. 221, 329-334 (1994).
60. O'Brien, M. A., Daily, W. J., Hesselberth, P. E., Moravec, R. A., Scurria, M. A., Klaubert, D. H., Bulleit, R. F., Wood, K. V. Homogeneous, bioluminescent protease assays: caspase-3 as a model. J. Biomol. Screening 10, 137-148 (2005).
61. Wehrman, T. S., von Dogenfeld, G., Krutzik, P. O., Nolan, G. P., Blau, H. M. Luminescent imaging of β-galactosidase activity in living subjects using sequential reporter-enzyme luminescence. Nat. Methods 3, 295-301 (2006).
62. Zhou, W., Valley, M. P., Shultz, J., Hawkins, E. M., Bernad, L., Good, T., Good, D., Riss, T. L., Klaubert, D. H., Wood, K. V. New bioluminogenic substrates for monoamine oxidase assays. J. Am. Chem. Soc. 128, 3122-3123 (2006).
63. Zhou, W., Shultz, J. W., Murphy, N., Hawkins, E. M., Bernad, L., Good, T., Moothart, L., Frackman, S., Klaubert, D. H., Bulleit, R. F., Wood, K. V. Electrophilic aromatic substituted luciferins as bioluminescent probes for glutathione S-transferase assays. Chem. Commun., 4620-4622 (2006).
64. Yao, H., So, M-k., Rao, J. A bioluminogenic substrate for in vivo imaging of β-lactamase activity. Angew. Chem. Int. Ed. 46, 7031-7034 (2007).
65. Zhou, W., Andrews, C., Liu, J., Shultz, J. W., Valley, M. P., Cali, J. J., Hawkins, Klaubert, D. H., Bulleit, R. F., Wood, K. V. Self-cleavable bioluminogenic luciferin phosphates as alkaline phosphatase reporters. ChemBioChem 9, 714-718 (2008).
66. Dragulescu-Andrasi, A., Liang, G., Rao, J. In vivo bioluminescence imaging of furin activity in breast cancer cells using bioluminogenic substrates. Bioconjugate Chem. 20, 1660-1666 (2009).
67. Jones, L. R., Goun, E. A., Shinde, R., Rothbard, J. B., Contag, C. H., Wender, P. A. Releasable luciferin-transporter conjugates: tools for the real-time analysis of cellular uptake and release. J. Am. Chem. Soc. 128, 6526-6527 (2006).
68. Wender, P. A., Goun, E. A., Jones, L. R., Pillow, T. H., Rothbard, J. B., Shinde, R., Contag, C. H. Real-time analysis of uptake and bioactivatable cleavage of luciferin-transporter conjugates in transgenic reporter mice. Proc. Natl. Acad. Sci. USA 104, 10340-10345 (2007).
69. Dubikovskaya, E. A., Thorne, S. H., Pillow, T. H., Contag, C. H., Wender, P. A. Overcoming multidrug resistance of small-molecule therapeutics through conjugation with releasable octaarginine transporters. Proc. Natl. Acad. Sci. USA 105, 12128-12133 (2008).
70. Denburg, J. L., Lee, R. T., McElroy, W. D. Substrate-binding properties of firefly luciferase: I. Luciferin-binding site. Arch. Biochem. Biophys. 134, 381-394 (1969).
71. Arnold, J. T. L., H.; McFann, K. K.; Blackman, M. R. Comparative effects of DHEA vs. testosterone, dihydrotestosterone, and estradiol on proliferation and gene expression in human LNCaP prostate cancer cells. Am. J. Physiol. Endocrinol. Metab. 288, E573-E584 (2005).
72. Antognelli, C., Del Buono, C., Baldracchini, F., Talesa, V., Cottini, E., Brancadoro, C., Zucchi, A., Mearini, E. Alteration of glyoxalases gene expression in response to testosterone in LNCaP and PC3 human prostate cancer cells. Cancer Biol. Ther. 6, 1880-1888 (2007).
73. Jennbacken, K., Gustaysson, H., Tesan, T., Horn, M., Vallbo, C., Welen, K., Damber, J.-E. The prostatic environment suppresses growth of androgen-independent prostate cancer xenografts: an effect influenced by testosterone. The Prostate 69, 1164-1175 (2009).
74. Sun, X.-Y. D., S. P.; Phang, J. M. Testosterone and prostate specific antigen stimulate generation of reactive oxygen species in prostate cancer cells. Carcinogenesis 22, 1775-1780 (2001).
75. Stewart, G. D. R., J. A.; McLaren, D. B.; Parker, C. C.; Habib, F. K.; Riddick, A. C. P. The relevance of a hypoxic tumour microenvironment in prostate cancer. BTU Int. 105, 8-13 (2009).
76. Lim, S. D. S., C.; Lambeth, J. D.; Marshall, F.; Amin, M.; Chung, L.; Petros, J. A.; Arnold, R. S. Increased NoxI and hydrogen peroxide in prostate cancer. The Prostate 62, 200-207 (2005).
77. Toya, Y. T., Masaharu; Kondo, Tadao; Nakata, Hisao; Isobe, Minoru; Goto, Toshio Improved synthetic methods of firefly luciferine derivatives for use in bioluminescent analysis of hydrolytic enzymes; carboxylic esterase and alkaline phosphatase. Bull. Chem. Soc. Jpn. 65, 2604-2610 (1992).
78. Branchini, B. R. et al. Anal. Biochem. 2010, 396, 290-296.
79. Mezzanotte, L. et al., In vivo bioluminescence imaging of murine xenograft cancer models with a red-shifted thermostable luciferase. Mol. Imaging. Biol. (2009, Nov. 9, online)

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A compound of the formula 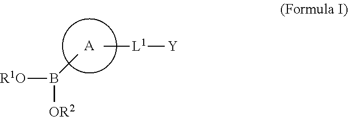
wherein R¹ and R² are selected from hydrogen and alkyl; or R¹ and R² together form a boronic ester ring or substituted boronic ester ring;
wherein A ring is selected from aryl, substituted aryl, heteroaryl, and substituted heteroaryl;
wherein L¹ is cleavable linker group that provides for release of Y upon reaction of the —B(OR¹)(OR²) group with a reactive oxygen species; and
wherein Y is a detectable moiety that is released upon reaction of the compound with a reactive oxygen species to generate a detectable signal.

2. The compound of claim 1, wherein —B(OR¹)(OR²) is selected from:

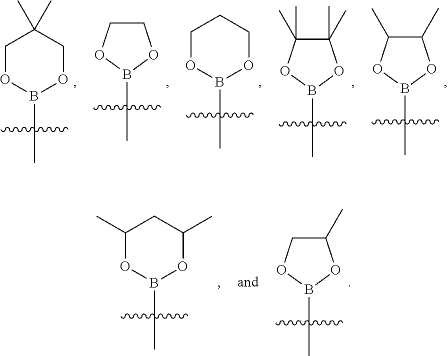

3. The compound of claim 1, wherein L¹ is described the following structure: 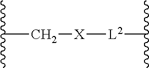
wherein X is a leaving group and L² is a linking group, wherein the bond that connects X to the adjacent —CH₂-group (CH₂—X) is a cleavable bond.

4. The compound of claim 1, wherein L¹ is selected from: 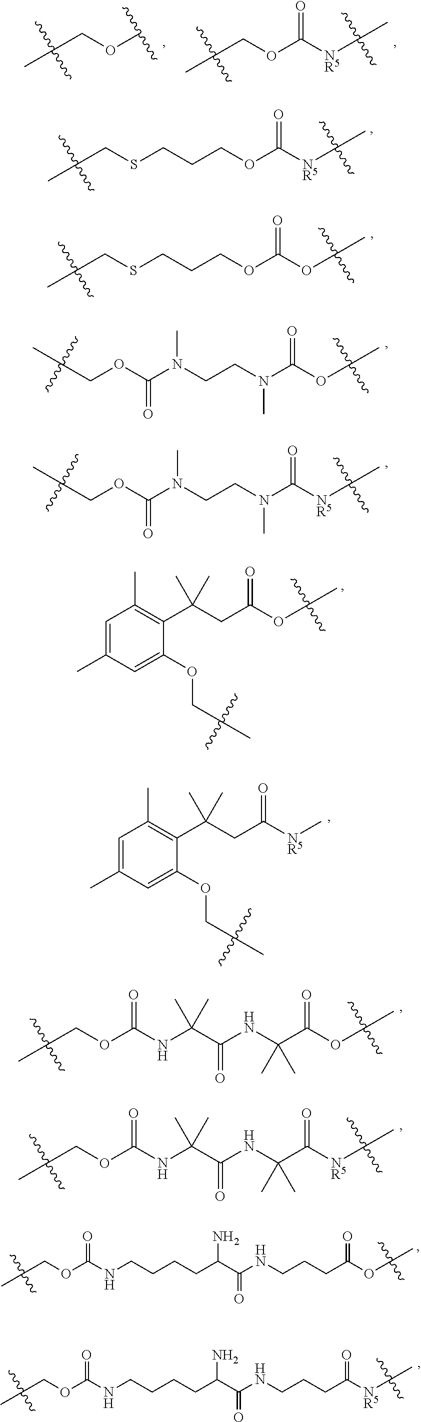 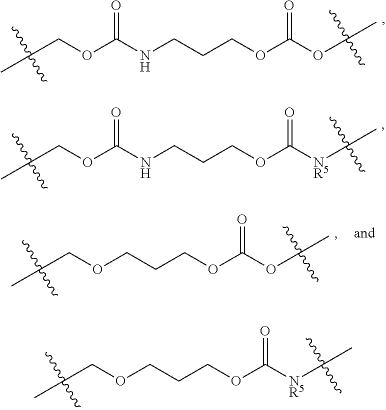
wherein R⁵ is hydrogen, an alkyl, a substituted alkyl or an alkoxy, wherein optionally R⁵ may be covalently connected to Y.

5. The compound of claim 1, wherein Y is luciferin, an aminoluciferin, a coelenterazine, a modified coelenterazine, a coelenterazine analog, dihydroluciferin, luciferin 6′ methylether, or luciferin 6′ chloroethylether.

6. The compound of claim 1, wherein the compound is of the formula: 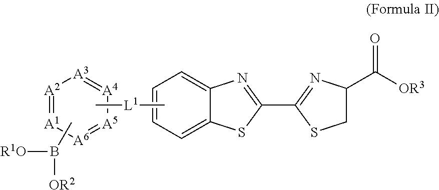
wherein R¹ and R² are selected from hydrogen and alkyl; or R¹ and R² together form a boronic ester ring or substituted boronic ester ring;
wherein each of A¹, A², A³, A⁴, A⁵, and A⁶ are independently selected from CH and N;
wherein L¹ is cleavable linker group that provides for release of the benzothiazolyl core upon reaction of the —B(OR¹)(OR²) group with a reactive oxygen species; and
wherein R³ is selected from hydrogen and alkyl.

7. The compound of claim 1, wherein the compound is of the formula: 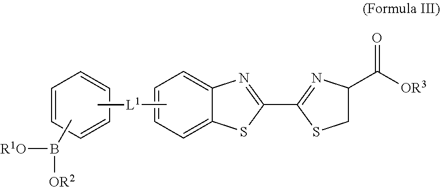
wherein R¹ and R² are selected from hydrogen and alkyl; or R¹ and R² together form a boronic ester ring or substituted boronic ester ring;
wherein L¹ is cleavable linker group that provides for release of the benzothiazolyl core upon reaction of the —B(OR¹)(OR²) group with a reactive oxygen species; and
wherein R³ is selected from hydrogen and alkyl.

8. The compound of claim 1, wherein the compound is of the formula: 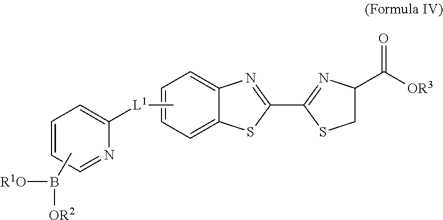
wherein R¹ and R² are selected from hydrogen and alkyl; or R¹ and R² together form a boronic ester ring or substituted boronic ester ring;
wherein L¹ is a cleavable linker group that provides for release of the benzothiazolyl core upon reaction of the —B(OR¹)(OR²) group with a reactive oxygen species; and
wherein R³ is selected from hydrogen and alkyl.

9. The compound of claim 1, wherein the compound is of the formula: 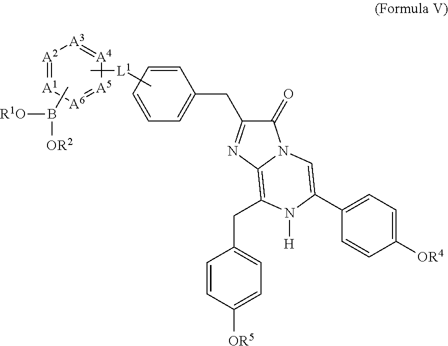
wherein R¹ and R² are selected from hydrogen and alkyl; or R¹ and R² together form a boronic ester ring or substituted boronic ester ring;
wherein each of A¹, A², A³, A⁴, A⁵, and A⁶ are independently selected from CH and N;
wherein L¹ is cleavable linker group that provides for release of the phenyl core upon reaction of the —B(OR¹)(OR²) group with a reactive oxygen species;
wherein R⁴ is selected from hydrogen and alkyl; and
wherein R⁵ is selected from hydrogen and alkyl.

10. The compound of claim 1, wherein the compound is of the formula: 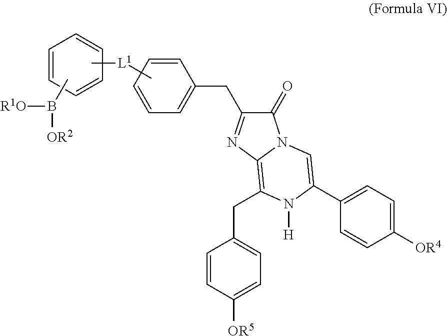
wherein R¹ and R² are selected from hydrogen and alkyl; or R¹ and R² together form a boronic ester ring or substituted boronic ester ring;
wherein L¹ is cleavable linker group that provides for release of the phenyl core upon reaction of the —B(OR¹)(OR²) group with a reactive oxygen species; and
wherein R⁴ is selected from hydrogen and alkyl; and
wherein R⁵ is selected from hydrogen and alkyl.

11. The compound of claim 1, wherein the compound is of the formula: 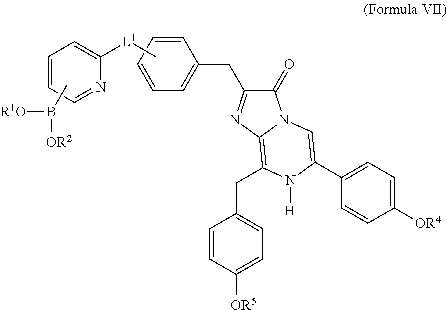
wherein R¹ and R² are selected from hydrogen and alkyl; or R¹ and R² together form a boronic ester ring or substituted boronic ester ring;
wherein L¹ is cleavable linker group that provides for release of the phenyl core upon reaction of the —B(OR¹)(OR²) group with a reactive oxygen species; and
wherein R⁴ is selected from hydrogen and alkyl; and
wherein R⁵ is selected from hydrogen and alkyl.

12. An optionally substituted coelenterazine derivative of formulae (IX), (X), or (XI): 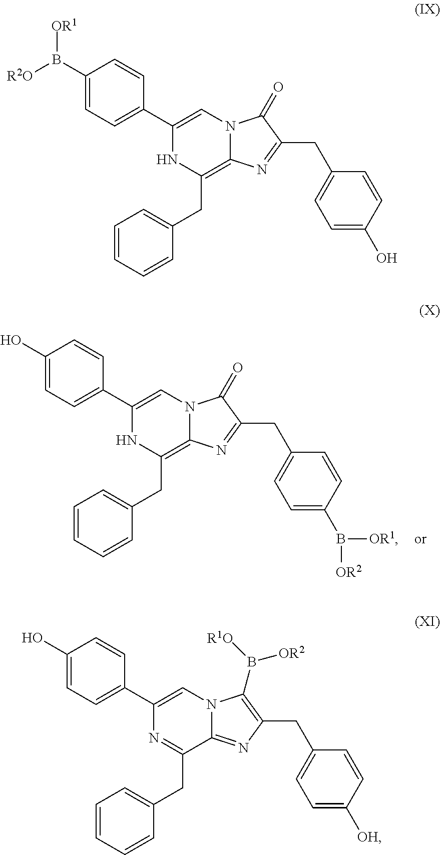
wherein R¹ and R² are selected from hydrogen and alkyl; or R¹ and R² together form a boronic ester ring or substituted boronic ester ring.

13. A composition comprising a compound of claim 1.

14. A method of detecting a reactive oxygen species (ROS) in a living cell, in a multicellular organism, or in a cell-free sample, the method comprising:
a) contacting the cell, the multicellular organism, or the cell-free sample with a compound of claim 1; and
b) detecting a signal produced by the compound upon reaction with the ROS.

15. The method of claim 14, wherein the ROS is hydrogen peroxide.

16. The method of claim 15, wherein hydrogen peroxide is selectively detected in a range of from about 2.5 μM to about 250 μM.

17. The method of claim 14, wherein the cell is in vitro.

18. The method of claim 14, wherein the cell is in a multicellular organism in vivo.

19. The method of claim 14, wherein the cell has been exposed to an internal or external stimulus.

20. The method of claim 14, wherein the cell is diseased.

21. The method of claim 14, wherein the cell is a mammalian cell.

22. The method of claim 14, wherein the ROS is in a cell in the multicellular organism.

23. The method of claim 22, wherein the ROS is present extracellularly in the multicellular organism.

24. The method of claim 14, wherein the multicellular organism is a mammal.
